(19) United States
(12) Patent Application Publication
(10) Pub. No.: US 2009/0085008 A1
(43) Pub. Date: Apr. 2, 2009

(54) Cellulose compound composition and cellulose compound film

(75) Inventors: Satoshi Tanaka, Kanagawa (JP); Shigeki Uehira, Kanagawa (JP); Nobutaka Fukagawa, Kanagawa (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Tokyo (JP)

(21) Appl. No.: 11/919,498

(22) PCT Filed: Oct. 24, 2006
(86) PCT No.: PCT/JP2006/321590

§ 371 Date: Oct. 29, 2007

(30) Foreign Application Priority Data
Oct. 24, 2005 (JP) 2005-309026

(51) Int. Cl. C07C 69/78; C07C 235/46; C09K 3/00

(52) U.S. Cl. 252/182.31; 560/60; 560/44; 564/153

(57) ABSTRACT

A cellulose compound composition, containing at least one compound of formula (1):

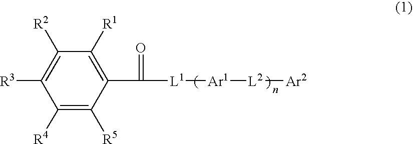

wherein R¹ to R⁵ are a hydrogen atom or a substituent, and any one of R¹ to R⁵ is -L³-Q¹, in which Q¹ is a group of formula Q1:

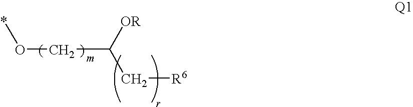

in which * is a bonding hand for bonding with L³, R is a hydrogen atom or an alkyl group, m and r are an integer of 0 or more, R⁶ is a hydrogen atom or a substituent; L¹, L² and L³ are a single bond or a divalent linking group, Ar¹ is an arylene or aromatic heterocyclic group, Ar² represents an aryl or aromatic heterocyclic group, and n is an integer of 0 or more, in which two or more L²s and two or more Ar¹s each may be the same or different from each other; and a film composed of the composition.

### TECHNICAL FIELD

The present invention relates to a cellulose compound composition and to a cellulose compound film composed of the cellulose compound composition.

### BACKGROUND ART

Among cellulose films, a cellulose acetate film has higher optical isotropy (a lower retardation value) than other polymer films. Therefore, it is common to use a cellulose acetate film in applications that require optical isotropy, e.g. for a polarizing plate. On the other hand, it is demanded that optical compensation sheets (retardation films (phase-contrast films, or phase difference films)) for use, for example, in a liquid crystal display device, have optical anisotropy (high retardation value), in reverse. Therefore, a synthetic polymer film, e.g. a polycarbonate film or polysulfone film, which has a high retardation value, is generally used as the optical compensation sheet.

Meanwhile, in recent years, there is proposed a cellulose acetate film which has such a high retardation value that it can also be used in applications that require optical anisotropy (see, for example, European Patent Application Publication (Laid-Open) No. 0911656A2, and JP-A-2003-344655 (“JP-A” means unexamined published Japanese patent application)). In the cellulose triacetate film, an aromatic compound having at least two aromatic rings, and particularly a compound having a 1,3,5-triazine ring, is added, and the resultant film is subjected to stretching, to attain a high retardation value. Further, as described in JP-A-12-95877, since a halogen-containing mixture solution (methylene chloride:methanol=87:13 (mass ratio)) is used as a solvent in the production of a cellulose acetate film, it is important that the compound to be added exhibits good solubility in the mixture solution.

Generally, the cellulose triacetate is a polymer material which can be stretched with difficulty, and it is difficult to sufficiently enhance its optical anisotropy. In the aforementioned European Patent Publication No. 0911656A2, an additive is oriented at the time when a film is stretched, to increase the optical anisotropy of the film, thereby accomplishing a high retardation value.

With regard to a liquid crystal display device in which the film is utilized, it is required that the device be lighter in mass and reduced in the production cost, and it is accordingly essential to develop a liquid crystal cell of a thinner film. In this regard, it is necessary for a film which is to be an optical compensation sheet to have high optical performances. The optical anisotropy (Re, a retardation value within the film surface; Rth, a retardation value in the direction of the thickness of the film), which can be attained in the compound film containing a 1,3,5-triazine ring, is still unsatisfactory, and it is required for a film to have a higher Re value and a lower Rth value. Specifically, the method disclosed in the aforementioned European Patent Publication No. 0911656A2 fails to make a film having a combination of desired Re and Rth values, and there is a need to develop novel technologies for controlling optical performances.

Further, taking reduction of the production cost into consideration, a compound which shows a high Re value and a low Rth value is wanted, even if it is used in a small amount to be added.

### DISCLOSURE OF INVENTION

The present invention contemplates providing a cellulose compound composition capable of forming a cellulose compound film having an optical anisotropy controlled and an excellent optical performance and of exhibiting good solubility in a halogen-containing mixture solution, and also providing a cellulose compound film composed of the cellulose compound composition.

According to the present invention, there is provided the following means:

[1] A cellulose compound composition, comprising at least one compound represented by formula (1):

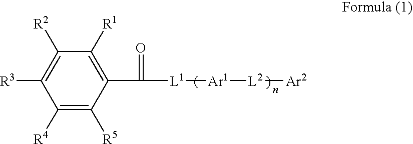

wherein R¹, R², R³, R⁴ and R⁵ each independently represent a hydrogen atom or a substituent, and any one of R¹, R², R³, R⁴ and R⁵ represents a group represented by -L³-Q¹, in which Q¹ represents a group represented by formula Q1:

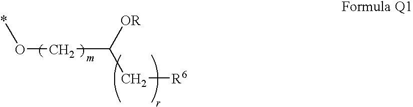

in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;

L¹, L² and L³ each independently represent a single bond or a divalent linking group, Ar¹ represents an arylene group or an aromatic heterocyclic group, Ar² represents an aryl group or an aromatic heterocyclic group, and n represents an integer of 0 or more, in which two or more L²s may be the same or different from each other and two or more Ar¹s may be the same or different from each other.

[2] The cellulose compound composition according to item [1], wherein R³ in formula (1) is the group represented by -L³-Q¹.
[3] The cellulose compound composition according to item [1] or [2], wherein the compound represented by formula (1) is a compound represented by formula (2):

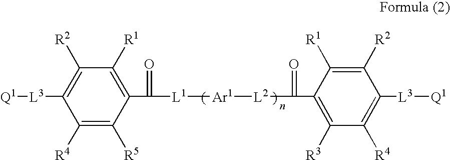

wherein R¹, R², R³, R⁴ and R⁵ each independently represent a hydrogen atom or a substituent, and Q¹ represents a group represented by formula Q1:

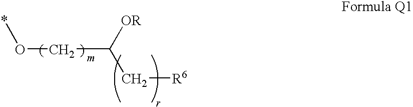

in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;

L¹, L² and L³ each independently represent a single bond or a divalent linking group, Ar¹ represents an arylene group or an aromatic heterocyclic group, and n represents an integer of 1 or more, in which two or more L²s may be the same or different from each other and two or more Ar¹s may be the same or different from each other.

[4] The cellulose compound composition according to any one of items [1] to [3], wherein a cellulose acylate is contained as the cellulose compound.
[5] A cellulose compound film, which is composed of the cellulose compound composition according to any one of items [1] to [4].
[6] A compound, represented by formula (1):

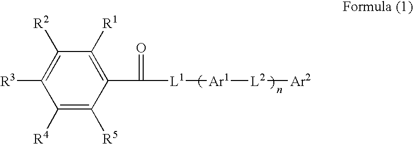

wherein R¹, R², R³, R⁴ and R⁵ each independently represent a hydrogen atom or a substituent, and any one of R¹, R², R³, R⁴ and R⁵ represents a group represented by -L³-Q¹, in which Q¹ represents a group represented by formula Q1:

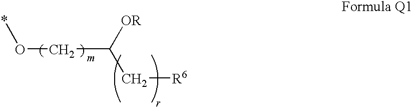

in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;

L¹, L² and L³ each independently represent a single bond or a divalent linking group, Ar¹ represents an arylene group or an aromatic heterocyclic group, Ar² represents an aryl group or an aromatic heterocyclic group, and n represents an integer of 0 or more, in which two or more L²s may be the same or different from each other and two or more Ar¹s may be the same or different from each other.

[7] A compound, represented by formula (2):

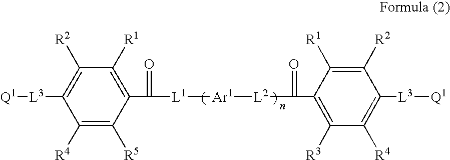

wherein , R¹, R², R³, R⁴ and R⁵ each independently represent a hydrogen atom or a substituent, and Q¹ represents a group represented by formula Q1:

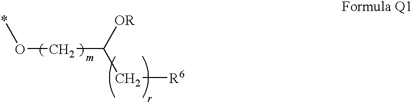

in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;

L¹, L² and L³ each independently represent a single bond or a divalent linking group, Ar¹ represents an arylene group or an aromatic heterocyclic group, and n represents an integer of 1 or more, in which two or more L²s may be the same or different from each other and two or more Ar¹s may be the same or different from each other.

Other and further features and advantages of the invention will appear more fully from the following description.

### BEST MODE FOR CARRYING OUT THE INVENTION

### Compound Represented by Formula (1) or (2)

First, the compound represented by formula (1), which is contained in the cellulose compound composition of the present invention, will be explained in detail:

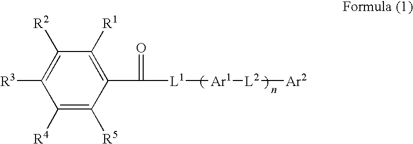

wherein R¹, R², R³, R⁴ and R⁵ each independently represent a hydrogen atom or a substituent, and any one of R¹, R², R³, R⁴ and R⁵ represents a group represented by -L³-Q¹, in which Q¹ represents a group represented by formula Q1:

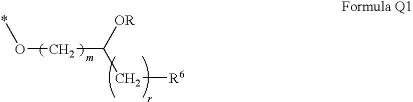

in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;

L¹, L² and L³ each independently represent a single bond or a divalent linking group, Ar¹ represents an arylene group or an aromatic heterocyclic group, Ar² represents an aryl group or an aromatic heterocyclic group, and n represents an integer of 0 or more, in which two or more L²s may be the same or different from each other and two or more Ar¹s may be the same or different from each other.

R¹, R², R³, R⁴ and R⁵ in formula (1), and R⁶ in formula Q1, each independently represent a hydrogen atom or a substituent. As the substituent, the substituent T which will be explained below may be applied. Any one of R¹, R², R³, R⁴ and R⁵ represents a group represented by -L³-Q¹, wherein Q¹ represents a group represented by formula Q1:

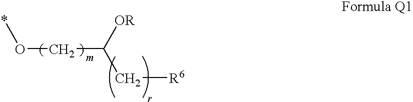

In formula Q1, * represents a bonding hand for bonding with L³, and the * represents a single bond or a divalent linking group. Examples of the divalent linking group include —O—, —NR′— (in which R′ represents a hydrogen atom, or an alkyl group or aryl group, which may have a substituent), —CO—, an alkylene group, a substituted alkylene group, an alkenylene group, a substituted alkenylene group, an alkinylene group, or a group obtained by combining two or more of these divalent groups. L³ is most preferably a single bond.

In formula Q1, R represents a hydrogen atom or an alkyl group having one or more carbon atoms. R is preferably an alkyl group having 1 to 8 carbon atoms, more preferably an alkyl group having 1 to 4 carbon atoms, and particularly preferably a methyl group.

In formula Q1, R⁶ is preferably a hydrogen atom, a halogen atom, an alkyl group, an alkoxy group, an amino group, a hydroxyl group, an acylamino group, or a sulfonylamino group, more preferably a hydrogen atom, a fluorine atom, a chlorine atom, an alkyl group having 1 to 4 carbon atoms, an alkoxy group having 1 to 12 carbon atoms, or a hydroxyl group, and particularly preferably an alkyl group (having preferably 1 to 12 carbon atoms, more preferably 1 to 8 carbon atoms; and further preferably 1 to 6 carbon atoms, and particularly preferably a methyl group).

In formula Q1, m denotes an integer of 0 or more, preferably 1 to 7, more preferably 2 to 7, and further preferably 2 or 3. In formula Q1, r denotes an integer of 0 or more, preferably 0 to 7, more preferably 0 to 4, and further preferably 0.

R¹ is preferably a hydrogen atom, a halogen atom, an alkyl group, an alkoxy group, an amino group, a hydroxyl group, an acylamino group, or a sulfonylamino group, more preferably a hydrogen atom, a fluorine atom, a chlorine atom, an alkyl group having 1 to 4 carbon. atoms, an alkoxy group having 1 to 12 carbon atoms, or a hydroxyl group, and particularly preferably an alkoxy group (having preferably 1 to 12 carbon atoms, more preferably 1 to 8 carbon atoms, and further preferably 1 to 6 carbon atoms, and particularly preferably a methoxy group), or a hydrogen atom.

R² is preferably a hydrogen atom, a halogen atom, an alkyl group, an alkoxy group, an amino group, an acylamino group, a sulfonylamino group, or a hydroxyl group, more preferably a hydrogen atom, an alkyl group, or an alkoxy group, and further preferably a hydrogen atom, an alkyl group (having preferably 1 to 4 carbon atoms, and more preferably a methyl group), or an alkoxy group (having preferably 1 to 12 carbon atoms, more preferably 1 to 8 carbon atoms, further preferably 1 to 6 carbon atoms, and particularly preferably 1 to 4 carbon atoms). R² is particularly preferably a hydrogen atom, a methyl group, or a methoxy group, and most preferably a hydrogen atom.

R³ is preferably a halogen atom, an alkyl group, an alkoxy group, or a group represented by -L³-Q¹, and more preferably a group represented by -L³-Q¹.

R⁴ is preferably a hydrogen atom, a halogen atom, an alkyl group, an alkoxy group, an amino group, or a hydroxyl group, more preferably a hydrogen atom, an alkyl group, or an alkoxy group, and further preferably a hydrogen atom, an alkyl group (having preferably 1 to 4 carbon atoms, and more preferably a methyl group), or an alkoxy group (having preferably 1 to 12 carbon atoms, more preferably 1 to 8 carbon atoms, further preferably 1 to 6 carbon atoms, and particularly preferably 1 to 4 carbon atoms). R⁴ is particularly preferably a hydrogen atom, a methyl group, or a methoxy group, and most preferably a hydrogen atom.

R⁵ is preferably a hydrogen atom, a halogen atom, an alkyl group, an alkoxy group, an amino group, or a hydroxyl group, more preferably a hydrogen atom, an alkyl group, or an alkoxy group, and further preferably a hydrogen atom, an alkyl group (having preferably 1 to 4 carbon atoms, and more preferably a methyl group), or an alkoxy group (having preferably 1 to 12 carbon atoms, more preferably 1 to 8 carbon atoms, further preferably 1 to 6 carbon atoms, and particularly preferably 1 to 4 carbon atoms). R⁵ is particularly preferably a hydrogen atom, a methyl group, or a methoxy group, and most preferably a hydrogen atom.

Ar¹ represents an arylene group or an aromatic heterocyclic group, and Ar¹s in the repeating unit represented by (Ar¹-L²)ₙ may be the same or different from each other. Ar² represents an aryl group or an aromatic heterocyclic group.

In formula (1), the arylene group represented by Ar¹ is preferably an arylene group having 6 to 30 carbon atoms, which may be a mono-ring or may form a condensed ring with another ring. Further, the arylene group may have a substituent, if possible. As the substituent, the substituent T, which will be described below, may be applied. The arylene group represented by Ar¹ has more preferably 6 to 20 carbon atoms, and particularly preferably 6 to 12 carbon atoms. Examples of the arylene group include a phenylene group, an o-methylphenylene group, and a naphthylene group.

In formula (1), the aryl group represented by Ar² is preferably an aryl group having 6 to 30 carbon atoms, which may be a mono-ring or may form a condensed ring with another ring. Further, the aryl group may have a substituent, if possible. As the substituent, the substituent T, which will be described below, may be applied. The aryl group represented by Ar² has more preferably 6 to 20 carbon atoms, and particularly preferably 6 to 12 carbon atoms. Examples of the aryl group include a phenyl group, and a naphthyl group.

The aromatic heterocyclic group represented by Ar¹ or Ar² in formula (1) may be an aromatic heterocyclic group containing at least one of an oxygen atom, a nitrogen atom and a sulfur atom, and is preferably a five- or six-membered aromatic heterocyclic group having at least one of an oxygen atom, a nitrogen atom and a sulfur atom. Specific examples of the aromatic heterocycle include furan, pyrrole, thiophene, imidazole, pyrazole, pyridine, pyradine, pyridazine, triazole, triazine, indole, indazole, purin, thiazoline, thiazole, thiadiazole, oxazoline, oxazole, oxadiazole, quinoline, isoquinoline, phthalazine, naphthyridine, quinoxaline, quinazoline, cinnoline, pteridine, acridine, phenanthroline, phenazine, tetrazole, benzimidazole, benzoxazole, benzothiazole, benzotriazole, tetrazaindene, pyrrolotriazole, and pyrazolotriazole. The aromatic heterocycle is preferably benzimidazole, benzoxazole, benzothiazole, or benzotriazole. Further, the aromatic heterocyclic group may have a substituent, if possible, and as the substituent, the substituent T, which will be described below, may be applied.

In formula (1), L¹ and L² each independently represent a single bond or a divalent linking group. L¹ and L² may be the same or different from each other. Further, L²s in the repeating unit represented by (Ar¹-L²)ₙ may be the same or different from each other.

Examples of the divalent linking group include —O—, —NR′— (in which R′ represents a hydrogen atom, or an alkyl group or aryl group, which may have a substituent), —CO—, —SO₂—, —S—, an alkylene group, a substituted alkylene group, an alkenylene group, a substituted alkenylene group, alkinylene group, or a group obtained by combining two or more of these divalent groups. R′ is preferably a hydrogen atom, an unsubstituted alkyl group having 1 to 12 carbon atoms, or an unsubstituted aryl group having 6 to 12 carbon atoms, more preferably a hydrogen atom or an unsubstituted alkyl group having 1 to 4 carbon atoms, and most preferably a hydrogen atom.

L¹ is more preferably —O—, —NR—, —NRSO₂—, —NRCO—, or —OCO—, and particularly preferably —O—. L² is more preferably a single bond, —O—, —CO—, —SO₂NR—, —NRSO₂—, —CONR—, —NRCO—, —COO—, —OCO—, or an alkinylene group, and most preferably a single bond, —CONR—, —NRCO—, —COO—, —OCO—, or an alkinylene group.

In the compound represented by formula (1) that can be used in the present invention, Ar¹ is linked to L¹ and L², and when Ar¹ is a phenylene group, the L¹ and L², or L² and L² each most preferably form a para position relationship (1,4-position relationship) in the -L¹-Ar¹-L²- and -L²-Ar¹-L²-.

In formula (1), n represents an integer of 0 or more, preferably of 1 to 7, more preferably of 2 to 7, and further preferably of 3 to 6.

Of the compounds represented by formula (1), particularly preferable are compounds, in which R³ is a group represented by -L³-Q¹; L¹ is —O— or —NR′—; L² is a single bond, —O—, —CO—, —NR′—, —SO₂NR′—, —NR′SO₂—, —CONR′—, —NR′CO—, —COO, —OCO—, or an alkinylene group; and n denotes 3 to 6 (in which R′ represents a hydrogen atom, or an alkyl or aryl group, which may have a substituent, and R′ is preferably a hydrogen atom).

The compound represented by formula (1) is preferably a compound represented by formula (2). Next, the compound represented by formula (2) will be explained:

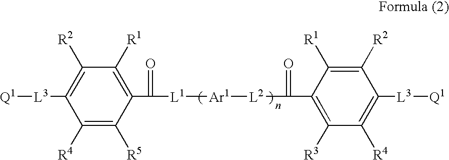

wherein R¹, R², R³, R⁴ and R⁵ each independently represent a hydrogen atom or a substituent, and Q¹ represents a group represented by formula Q1:

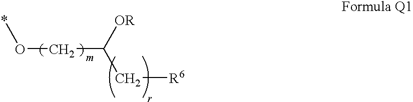

in which * represents a bonding hand for bonding with L 3, R represents a hydrogen atom or an alkyl group having one or more carbon atoms, m denotes an integer of 0 or more, r represents an integer of 0 or more, R⁶ represents a hydrogen atom or a substituent;

L¹, L² and L³ each independently represent a single bond or a divalent linking group, Ar¹ represents an arylene group or an aromatic heterocyclic group, and n represents an integer of 1 or more, in which two or more L²s may be the same or different from each other and two or more Ar¹s may be the same or different from each other.

R¹, R², R³, R⁴, R⁵, L¹, L², L³ and Q¹ in formula (2) have the same meanings as those explained in the above, and the preferable ranges are also the same, respectively.

n denotes an integer of 1 or more, preferably 1 to 7, more preferably 2 to 7, and further preferably 3 to 6.

Preferable examples of the compound represented by formula (2) include those, in which L³ is a single bond, —CO—, or —OCO—; R is a methyl group; m is 2 or 3; r is 0; R⁶ is a methyl group; L¹ is —O— or —NR′— (in which R′ represents a hydrogen atom, or an alkyl or aryl group, which may have a substituent, and R′ is preferably a hydrogen atom); L² is a single bond, —O—, —CO—, —NR′—, —SO₂NR′—, —NR′SO₂—, —CONR′—, —NR′CO—, —COO—, —OCO—, or an alkinylene group (in which R′ represents a hydrogen atom, or an alkyl or aryl group, which may have a substituent, and R′ is preferably a hydrogen atom); Ar¹ is an arylene group; and n is 3 to 6.

The aforementioned substituent T is explained below.

Preferable examples of the substituent T include a halogen atom (e.g., a fluorine atom, a chlorine atom, a bromine atom, an iodine atom), an alkyl group (preferably an alkyl group having 1 to 30 carbon atoms, e.g., a methyl group, an ethyl group, a n-propyl group, an isopropyl group, a t-butyl group, a n-octyl group, a 2-ethylhexyl group), a cycloalkyl group (preferably a substituted or unsubstituted cycloalkyl group having 3 to 30 carbon atoms, e.g., a cyclohexyl group, a cyclopentyl group, a 4-n-dodecylcyclohexyl group), a bicycloalkyl group (preferably a substituted or unsubstituted bicycloalkyl group having 5 to 30 carbon atoms, that is, a monovalent group obtained by removing one hydrogen atom from a bicycloalkane having 5 to 30 carbon atoms, e.g., a bicyclo[1,2,2]heptane-2-yl group, a bicyclo[2,2,2]octane-3-yl group), an alkenyl group (preferably a substituted or unsubstituted alkenyl group having 2 to 30 carbon atoms, e.g., a vinyl group, an allyl group), a cycloalkenyl group (preferably a substituted or unsubstituted cycloalkenyl group having 3 to 30 carbon atoms, that is, a monovalent group obtained by removing one hydrogen atom from a cycloalkene having 3 to 30 carbon atoms, e.g., a 2-cyclopentene-1-yl group, a 2-cyclohexene-1-yl group), a bicycloalkenyl group (a substituted or unsubstituted bicycloalkenyl group, preferably a substituted or unsubstituted bicycloalkenyl group having 5 to 30 carbon atoms, that is, a monovalent group obtained by removing one hydrogen atom from a bicycloalkene having one double bond, e.g., a bicyclo[2,2,1]hepto-2-ene-1-yl group, a bicyclo[2,2,2]octo-2-ene-4-yl group), an alkynyl group (preferably a substituted or unsubstituted alkynyl group having 2 to 30 carbon atoms, e.g., an ethynyl group, a propargyl group), an aryl group (preferably a substituted or unsubstituted aryl group having 6 to 30 carbon atoms, e.g., a phenyl group, a p-tolyl group, a naphthyl group), a heterocyclic group (preferably a 5- or 6-membered substituted or unsubstituted heterocyclic group, that is a monovalent group obtained by removing one hydrogen atom from an aromatic or non-aromatic heterocyclic compound, more preferably a 5- or 6-membered aromatic heterocyclic group having 3 to 30 carbon atoms, e.g., a 2-furyl group, a 2-thienyl group, a 2-pyrimidinyl group, a 2-benzothiazolyl group), a cyano group, a hydroxyl group, a nitro group, a carboxyl group, an alkoxy group (preferably a substituted or unsubstituted alkoxy group having 1 to 30 carbon atoms, e.g., a methoxy group, an ethoxy group, an isopropoxy group, a t-butoxy group, a n-octyloxy group, a 2-methoxyethoxy group), an aryloxy group (preferably a substituted or unsubstituted aryloxy group having 6 to 30 carbon atoms, e.g., a phenoxy group, a 2-methylphenoxy group, a 4-tert-buthylphenoxy group, a 3-nitrophenoxy group, a 2-tetradecanoylaminophenoxy group), a silyloxy group (preferably a silyloxy group having 3 to 20 carbon atoms, e.g., a trimethylsilyloxy group, a tert-butyldimethylsilyloxy group), a heterocyclic oxy group (preferably a substituted or unsubstituted heterocyclic oxy group having 2 to 30 carbon atoms, e.g., a 1-phenyltetrazole-5-oxy group, a 2-tetrahydropyranyloxy group), an acyloxy group (preferably a formyloxy group, a substituted or unsubstituted alkylcarbonyloxy group having 2 to 30 carbon atoms, and a substituted or unsubstituted arylcarbonyloxy group having 6 to 30 carbon atoms, e.g., a formyloxy group, an acetyloxy group, a pivaloyloxy group, a stealoyloxy group, a benzoyloxy group, a p-methoxyphenylcarbonyloxy group), a carbamoyloxy group (preferably a substituted or unsubstituted carbamoyloxy group having 1 to 30 carbon atoms, e.g., an N,N-dimethylcarbamoyloxy group, an N,N-diethylcarbamoyloxy group, a morpholinocarbonyloxy group, an N,N-di-n-octylaminocarbonyloxy group, an N-n-octylcarbamoyloxy group), an alkoxycarbonyloxy group (preferably a substituted or unsubstituted alkoxycarbonyloxy group having 2 to 30 carbon atoms, e.g., a methoxycarbonyloxy group, an ethoxycarbonyloxy group, a tert-butoxycarbonyloxy group, a n-octylcarbonyloxy group), an aryloxycarbonyloxy group (preferably a substituted or unsubstituted aryloxycarbonyloxy group having 7 to 30 carbon atoms, e.g., a phenoxycarbonyloxy group, a p-methoxyphenoxycarbonyloxy group, a p-n-hexadecyloxyphenoxycarbonyloxy group), an amino group (preferably an amino group, a substituted or unsubstituted alkylamino group having 1 to 30 carbon atoms, and a substituted or unsubstituted anilino group having 6 to 30 carbon atoms, e.g., an amino group, a methylamino group, a dimethylamino group, an anilino group, an N-methyl-anilino group, a diphenylamino group), an acylamino group (preferably a formylamino group, a substituted or unsubstituted alkylcarbonylamino group having 1 to 30 carbon atoms, and a substituted or unsubstituted arylcarbonylamino group having 6 to 30 carbon atoms, e.g., a formylamino group, an acetylamino group, a pivaloylamino group, a lauroylamino group, a benzoylamino group), an aminocarbonylamino group (preferably a substituted or unsubstituted aminocarbonylamino group having 1 to 30 carbon atoms, e.g., a carbamoylamino group, an N,N-dimethylaminocarbonylamino group, an N,N-diethylaminocarbonylamino group, a morpholinocarbonylamino group), an alkoxycarbonylamino group (preferably a substituted or unsubstituted alkoxycarbonylamino group having 2 to 30 carbon atoms, e.g., a methoxycarbonylamino group, an ethoxycarbonylamino group, a tert-butoxycarbonylamino group, a n-octadecyloxycarbonylamino group, an N-methyl-methoxycarbonylamino group), an aryloxycarbonylamino group (preferably a substituted or unsubstituted aryloxycarbonylamino group having 7 to 30 carbon atoms, e.g., a phenoxycarbonylamino group, a p-chlorophenoxycarbonylamino group, a m-n-octyloxyphenoxycarbonylamino group), a sulfamoyl amino group (preferably a substituted or unsubstituted sulfamoylamino group having 0 (zero) to 30 carbon atoms, e.g., a sulfamoylamino group, an N,N-dimethylaminosulfonylamino group, an N-n-octylaminosulfonylamino group), an alkyl- or aryl-sulfonylamino group (preferably a substituted or unsubstituted alkylsulfonylamino group having 1 to 30 carbon atoms, a substituted or unsubstituted arylsulfonylamino group having 6 to 30 carbon atoms, e.g., a methylsulfonylamino group, a butylsulfonylamino group, a phenylsulfonylamino group, a 2,3,5-trichlorophenylsulfonylamino group, a p-methylphenylsulfonylamino group), a mercapto group, an alkylthio group (preferably a substituted or unsubstituted alkylthio group having 1 to 30 carbon atoms, e.g., a methylthio group, an ethylthio group, a n-hexadecylthio group), an arylthio group (preferably a substituted or unsubstituted arylthio group having 6 to 30 carbon atoms, e.g., a phenylthio group, a p-chlorophenylthio group, a m-methoxyphenylthio group), a heterocyclic thio group (preferably a substituted or unsubstituted heterocyclic thio group having 2 to 30 carbon atoms, e.g., a 2-benzothiazolylthio group, a 1-phenyltetrazol-5-yl thio group), a sulfamoyl group (preferably a substituted or unsubstituted sulfamoyl group having 0 (zero) to 30 carbon atoms, e.g., an N-ethylsulfamoyl group, an N-(3-dodecyloxypropyl)sulfamoyl group, an N,N-dimethylsulfamoyl group, an N-acetylsulfamoyl group, an N-benzoylsulfamoyl group, an N—(N′-phenylcarbamoyl)sulfamoyl group), a sulfo group, an alkyl- or aryl-sulfinyl group (preferably a substituted or unsubstituted alkylsulfinyl group having 1 to 30 carbon atoms, or a substituted or unsubstituted arylsulfinyl group having 6 to 30 carbon atoms, e.g., a methylsulfinyl group, an ethylsulfinyl group, a phenylsulfinyl group, a p-methylphenylsulfinyl group), an alkyl- or aryl-sulfonyl group (preferably a substituted or unsubstituted alkylsulfonyl group having 1 to 30 carbon atoms, or a substituted or unsubstituted arylsulfonyl group having 6 to 30 carbon atoms, e.g., a methylsulfonyl group, an ethylsulfonyl group, a phenylsulfonyl group, a p-methylphenylsulfonyl group), an acyl group (preferably a formyl group, a substituted or unsubstituted alkylcarbonyl group having 2 to 30 carbon atoms, or a substituted or unsubstituted arylcarbonyl group having 7 to 30 carbon atoms, e.g., an acetyl group, a pivaloylbenzoyl group), an aryloxycarbonyl group (preferably a substituted or unsubstituted aryloxycarbonyl group having 7 to 30 carbon atoms, e.g., a phenoxycarbonyl group, an o-chlorophenoxycarbonyl group, a m-nitrophenoxycarbonyl group, a p-tert-butylphenoxycarbonyl group), an alkoxycarbonyl group (preferably a substituted or unsubstituted alkoxycarbonyl group having 2 to 30 carbon atoms, e.g., a methoxycarbonyl group, an ethoxycarbonyl group, a tert-butoxycarbonyl group, a n-octadecyloxycarbonyl group), a carbamoyl group (preferably a substituted or unsubstituted carbamoyl group having 1 to 30 carbon atoms, e.g., a carbamoyl group, an N-methylcarbamoyl group, an N,N-dimethylcarbamoyl group, an N,N-di-n-octylcarbamoyl group, an N-(methylsulfonyl)carbamoyl group), an aryl- or heterocyclic-azo group (preferably a substituted or unsubstituted arylazo group having 6 to 30 carbon atoms or a substituted or unsubstituted heterocyclic azo group having 3 to 30 carbon atoms, e.g., a phenylazo group, a p-chlorophenylazo group, a 5-ethylthio-1,3,4-thiadiazole-2-yl azo group), an imido group (preferably an N-succinimido group, an N-phthalimido group), a phosphino group (preferably a substituted or unsubstituted phosphino group having 2 to 30 carbon atoms, e.g., a dimethylphosphino group, a diphenylphosphino group, a methylphenoxyphosphino group), a phosphinyl group (preferably a substituted or unsubstituted phosphinyl group having 2 to 30 carbon atoms, e.g., a phosphinyl group, a dioctyloxyphosphinyl group, a diethoxyphosphinyl group), a phosphinyloxy group (preferably a substituted or unsubstituted phosphinyloxy group having 2 to 30 carbon atoms, e.g., a diphenoxyphosphinyloxy group, a dioctyloxyphosphinyloxy group), a phosphinylamino group (preferably a substituted or unsubstituted phosphinylamino group having 2 to 30 carbon atoms, e.g., a dimethoxyphosphinylamino group, a dimethylaminophosphinylamino group), and a silyl group (preferably a substituted or unsubstituted silyl group having 3 to 30 carbon atoms, e.g., a trimethylsilyl group, a tert-butyldimethylsilyl group, a phenyldimethylsilyl group).

Of the above-mentioned substituents, those substituents which have hydrogen atom(s) may be further substituted with the above groups in place of the hydrogen atom(s). Examples of such functional groups include an alkylcarbonylaminosulfonyl group, an arylcarbonylaminosulfonyl group, an alkylsulfonylaminocarbonyl group, and an arylsulfonylaminocarbonyl group. Examples thereof include methylsulfonylaminocarbonyl, p-methylphenylsulfonylaminocarbonyl, acetylaminosulfonyl, and benzoylaminosulfonyl.

When there are two or more substituents, they may be the same or different. The substituents may bond together, to form a ring, if possible.

Specific examples of the compound represented by formula (1) or (2) are shown below, but the invention is not meant to be limited to those.

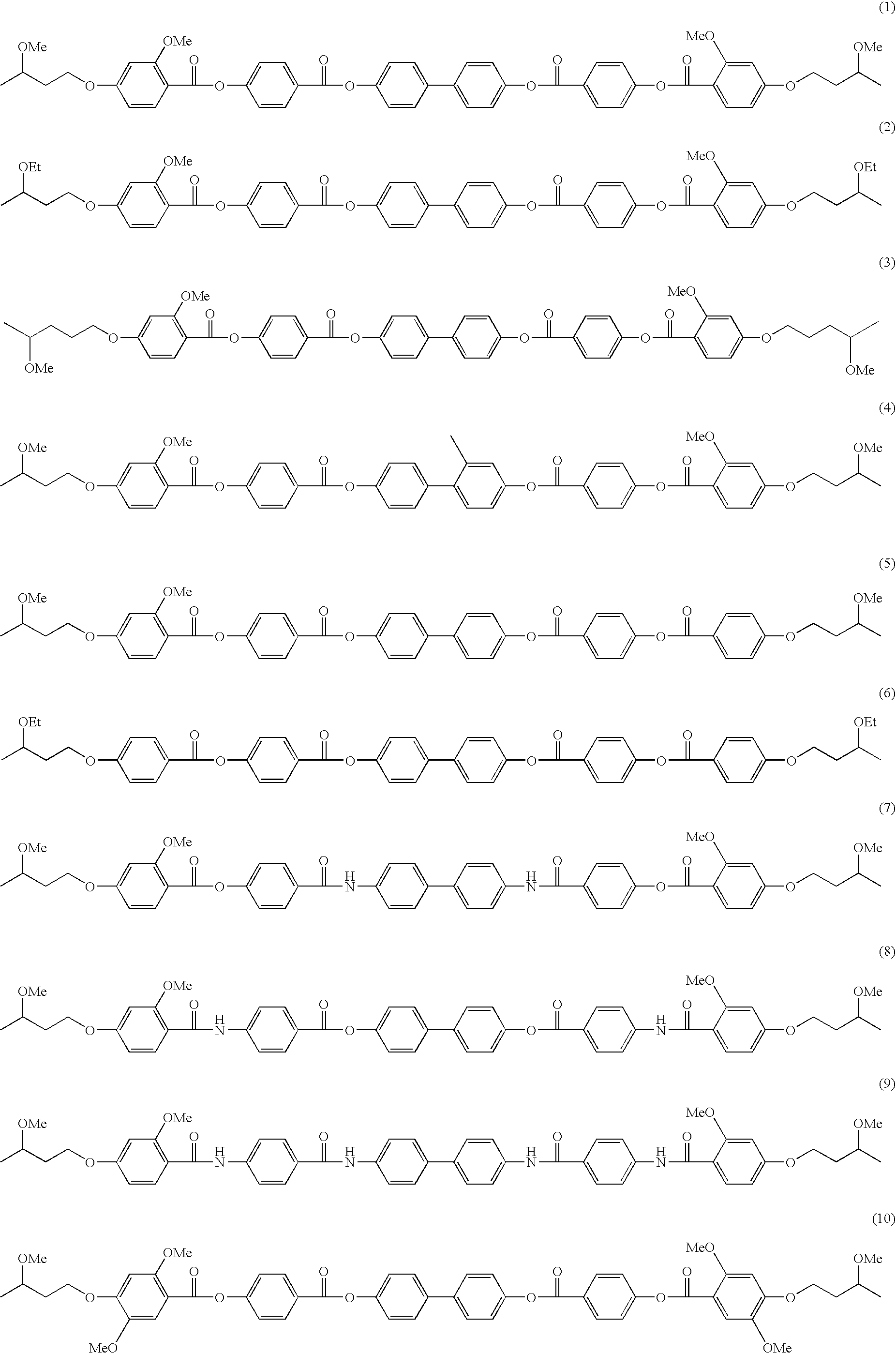

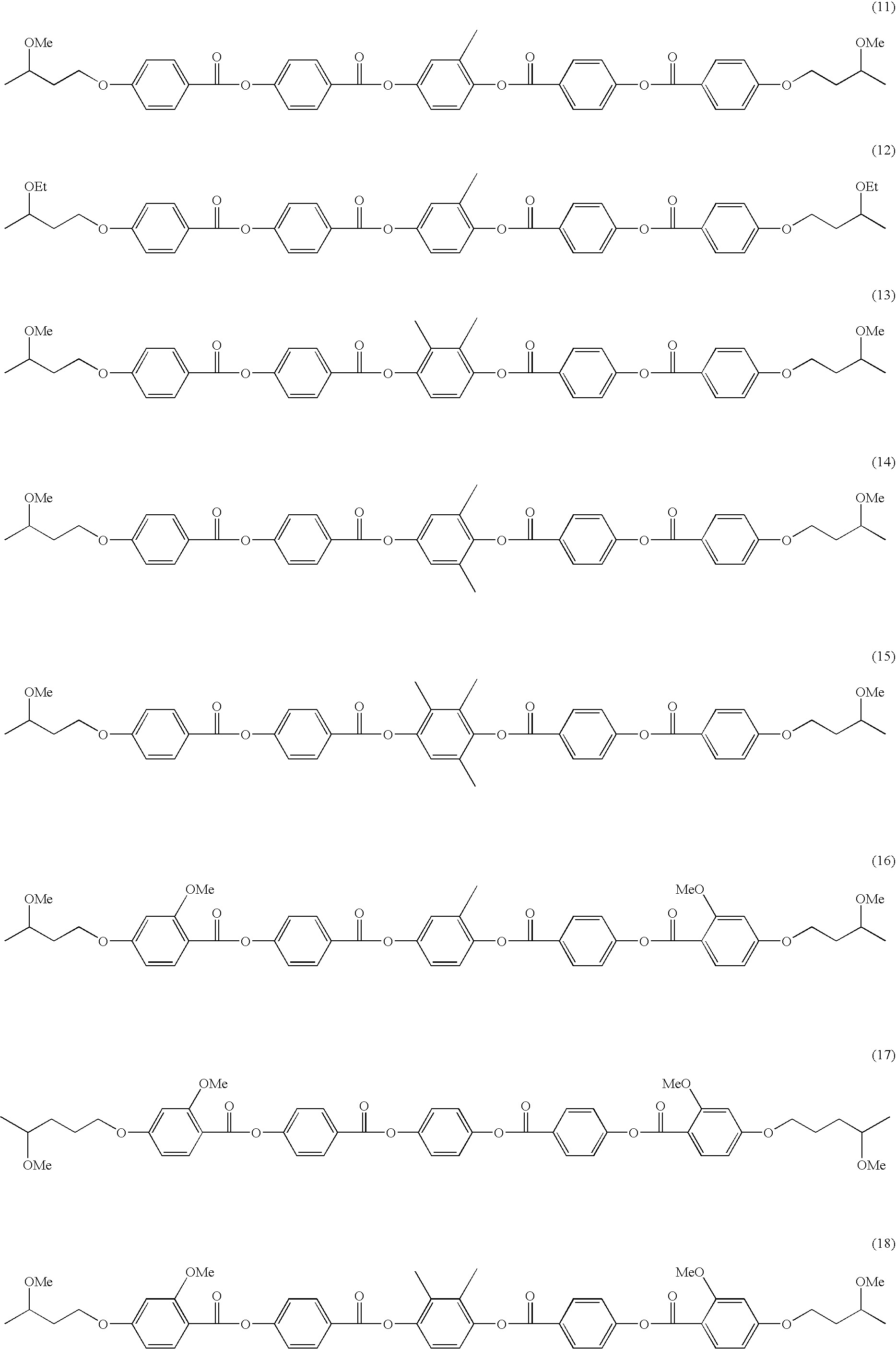

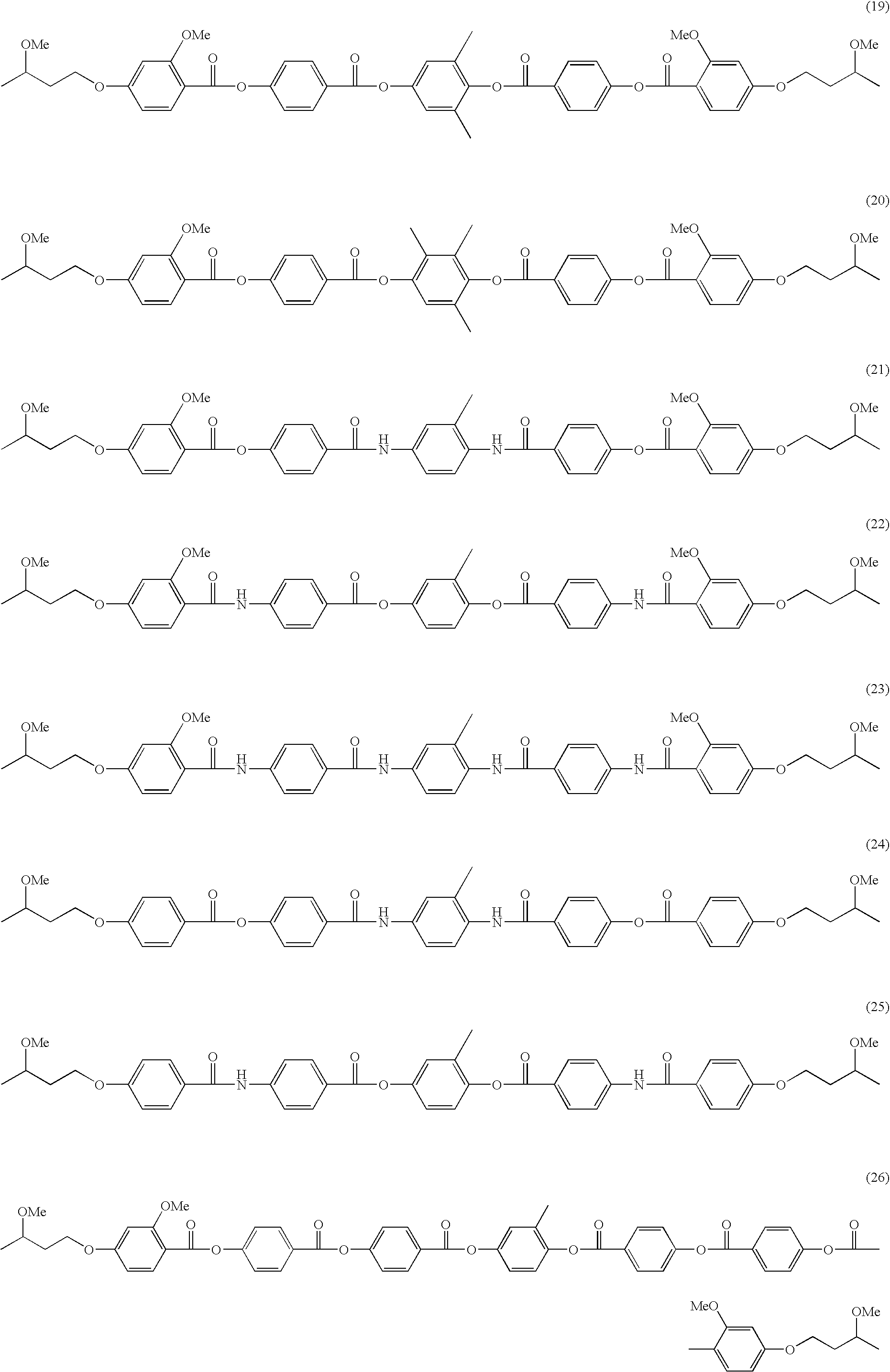

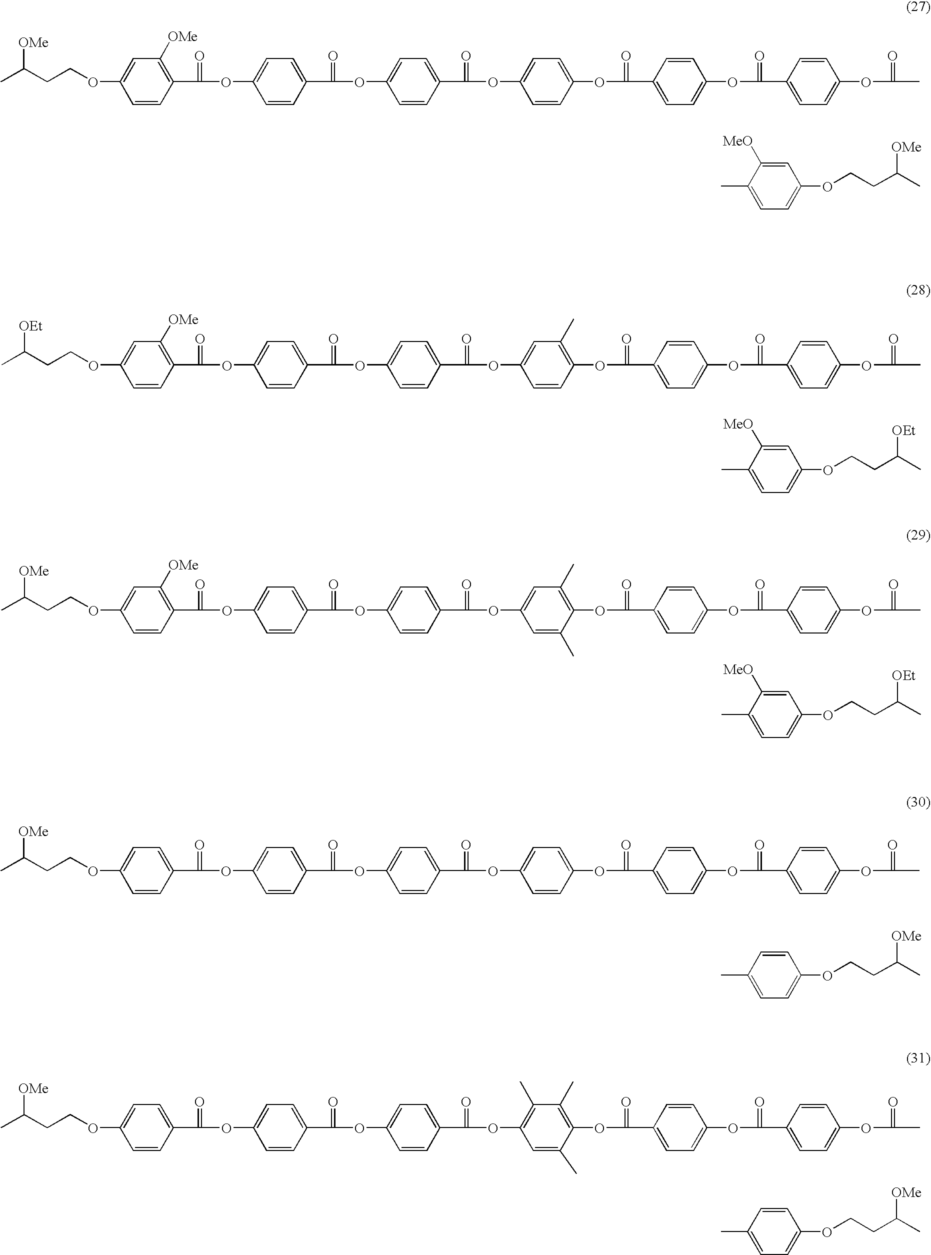

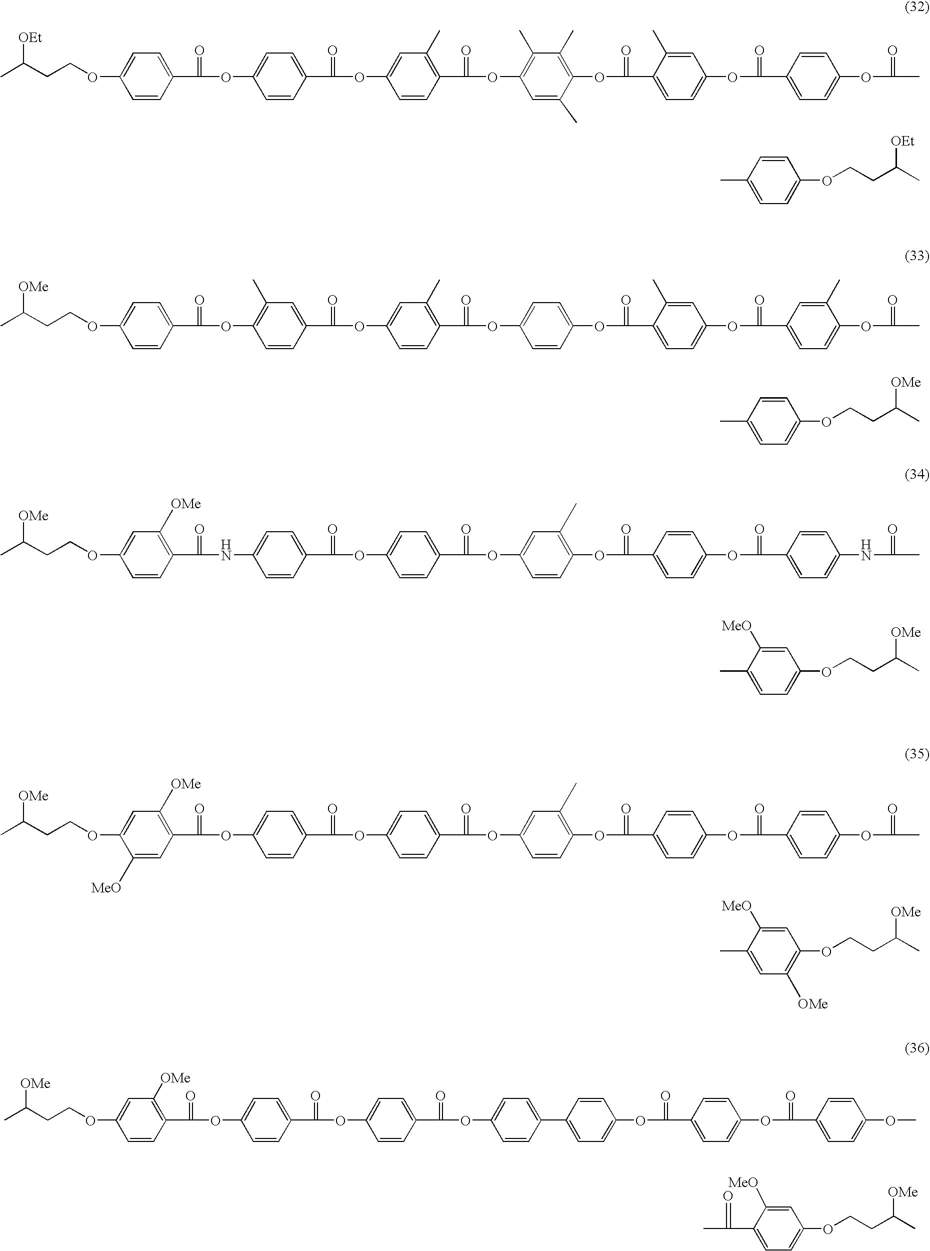

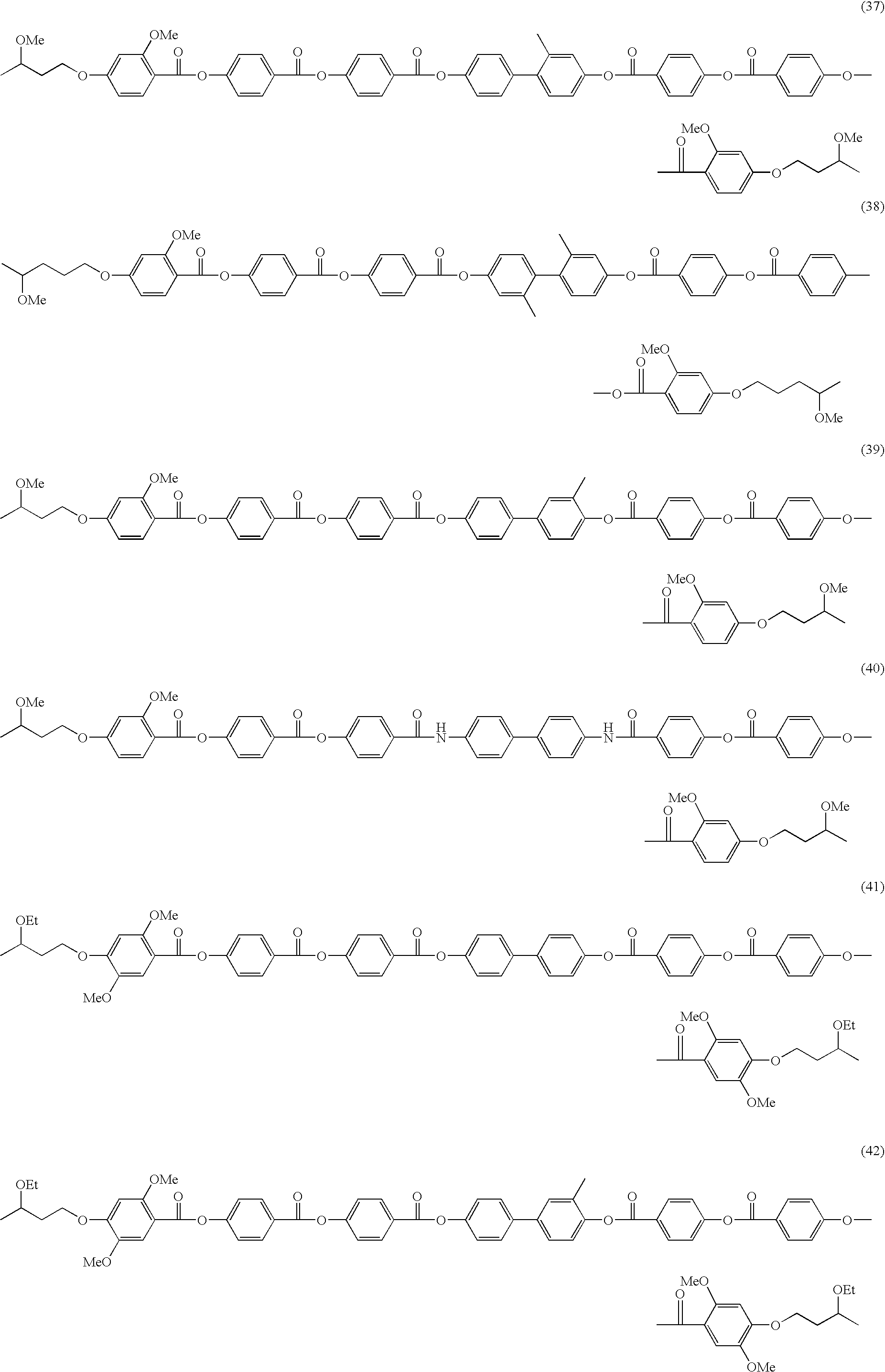

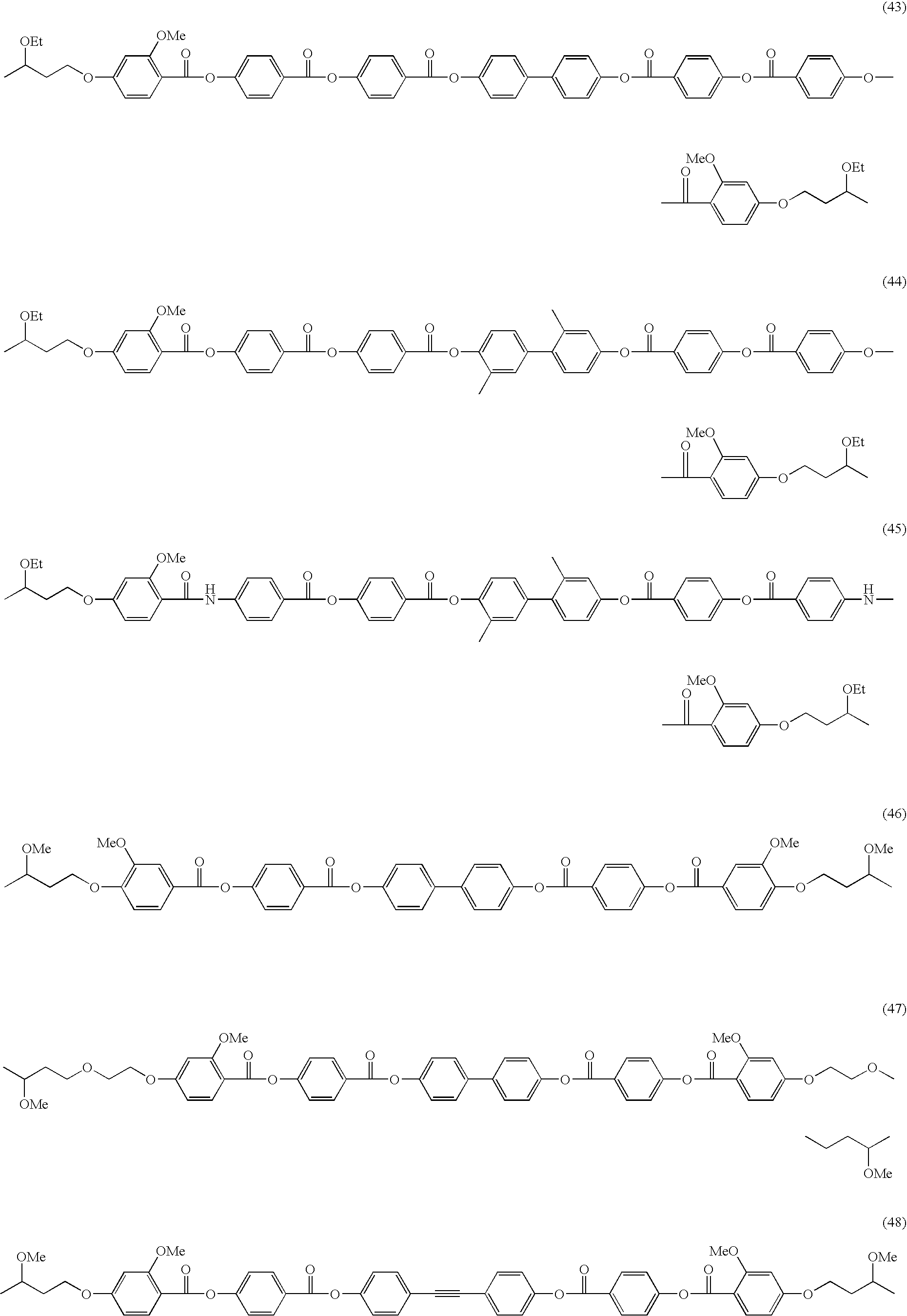

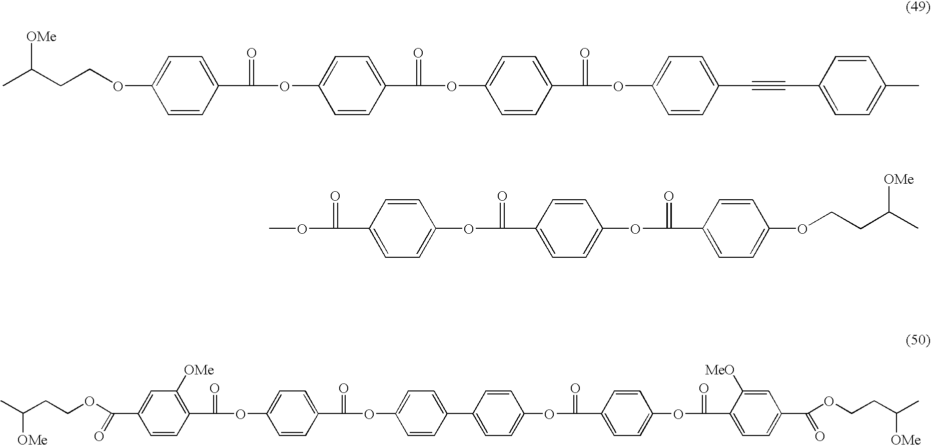

The compound represented by formula (1) or (2) can be produced, with reference to any known synthetic method.

In the cellulose compound composition of the present invention, at least one of the compound represented by formula (1) or (2) is added in an amount of preferably 0.1 to 20% by mass, more preferably 0.5 to 16% by mass, further preferably 1 to 12% by mass, still further preferably 1 to 8% by mass, and particularly preferably 1 to 5% by mass, to the cellulose compound.

### [Cellulose Compound Composition]

The cellulose compound composition of the present invention is a cellulose compound composition comprising: at least one cellulose compound; and at least one selected from the compound represented by formula (1) or (2). In the present invention, the cellulose compound means cellulose, or a compound having a cellulose skeleton obtained by introducing a functional group biologically or chemically into a cellulose to be used as a raw material. The cellulose compound is preferably a cellulose ester, and more preferably a cellulose acylate (examples thereof include a cellulose triacylate and a cellulose acylate propionate). Further, in the present invention, two or more cellulose acylates different from each other may be blended to use.

In the present invention, the cellulose compound composition may be a liquid (for example, a cellulose compound solution), or a solid (for example, a cellulose compound film).

The cellulose compound that can be used in the cellulose compound composition of the present invention is preferably a cellulose acylate, as mentioned above. A preferred embodiment of the cellulose compound composition of the present invention will be explained below, in which a cellulose acylate is to be an exemplified example.

### [Cellulose Acylate Raw Cotton]

Examples of the cellulose of the cellulose acylate raw material include cotton linter and wood pulp (broadleaf pulp, and conifer (needleleaf) pulp). Any cellulose acetate obtained from any raw cellulose may be used, and a plurality of celluloses may be used in combination of two or more thereof according to the need. There are detailed descriptions of these raw celluloses in, for example, “Plastic Material Lectures (17) Cellulose Resin” (Marusawa and Uda, The Nikkan Kogyo Shimbun, Ltd., published in 1970); and Japan Institute of Invention and Innovation, Kokai Giho (Open Technical Report) 2001-1745 (pp. 7 to 8), and the raw celluloses described in these publications may be used in the present invention, but these examples are not intended to be limiting of the invention.

The aforementioned cellulose acylate is preferably a mixed fatty acid ester of a cellulose obtained by substituting a hydroxyl group with an acetyl group and a cellulose obtained by substituting a hydroxyl group with an acyl group having 3 or more carbon atoms, and the degree of substitution for a hydroxyl group of the cellulose satisfies the following expressions (4) and (5).

2.0≦A+B≦3.0 Expression (4)

0<B Expression (5)

Herein, A and B in the expressions represent the degree of substitution of an acyl group substituting for a hydroxyl group of the cellulose, wherein A represents the degree of substitution of an acetyl group, and B represents the degree of substitution of an acyl group having 3 or more carbon atoms.

Each of the glucose units, which constitute cellulose by bonding through β-1,4-glycoside bond, has free hydroxyl groups at the 2-, 3-, and 6-positions thereof. A cellulose acylate is a polymer obtained by esterifying a part or the whole of these hydroxyl groups with an acyl group(s). Herein, the “substitution degree” means the ratio of esterification at the 2-, 3-, or 6-positions in the cellulose. Specifically, the 100% esterification of any one of the 2-, 3-, and 6-positions is a substitution degree of 1.

### [Degree of Polymerization of Cellulose Acylate]

The degree of polymerization of cellulose acylate is preferably 180 to 700 in terms of viscosity average degree of polymerization. In the case of cellulose acetate, the degree of polymerization is preferably 180 to 550, more preferably 180 to 400, and particularly preferably 180 to 350, in terms of viscosity average degree of polymerization. If the degree of polymerization is too large, the viscosity of a dope solution of cellulose acylate becomes high, which makes it difficult to produce a film by casting. When the degree of polymerization is too low, the mechanical strength of the produced film becomes low. The average polymerization degree can be measured by a limiting viscosity method by Uda et al., (Kazuo Uda and Hideo Saito, “The Journal of the Society of Fiber Science and Technology, Japan”, Vol. 18, No. 1, pp. 105 to 120, 1962). The method is also described in detail in JP-A-9-95538.

Further, the distribution of molecular weight of a cellulose acylate is evaluated by gel permeation chromatography. It is preferable that the polydisperse index Mw/Mn (Mw, mass average molecular weight; and Mn, number average molecular weight) be small and the distribution of molecular weight be narrow. Specifically, the value of Mw/Mn is preferably from 1.0 to 3.0, more preferably from 1.0 to 2.0, and particularly preferably from 1.0 to 1.6.

If a low molecular weight component(s) is removed from the cellulose acylate, the average molecular weight (polymerization degree) thereof becomes higher, but the viscosity thereof becomes lower than that of ordinary cellulose acylate, which means that the removal is useful. A cellulose acylate containing a low molecular weight component(s) at a small ratio can be obtained by removing the low molecular weight component(s) from a cellulose acylate synthesized in a usual manner. The removal of the low molecular weight component(s) can be carried out by washing the cellulose acylate with an appropriate organic solvent. When the cellulose acylate containing a small amount of the low molecular weight component(s) is to be produced, the amount of a sulfuric acid catalyst in the acetylation reaction is preferably adjusted to 0.5 to 25 parts by mass to 100 parts by mass of the cellulose. When the amount of the sulfuric acid catalyst is set within the range, a cellulose acylate having a preferable molecular weight distribution (uniform molecular weight distribution) can be synthesized. In the case that the cellulose acylate is used when the cellulose acylate film of the present invention is produced, the percentage of water content in the cellulose acylate is preferably 2 mass % or less, more preferably 1 mass % or less, particularly preferably 0.7 mass % or less. It is known that cellulose acylate generally contains water in an amount of 2.5 to 5% by mass. Thus, in order to set the percentage of water content in the cellulose acylate within the range, it is necessary to dry ordinary cellulose acylate. The method for the drying is not particularly limited as far as the target percentage of water content can be attained. About the cellulose acylate used in the present invention, the starting cotton thereof, and the synthesis method thereof are described in detail in “Kokai Giho” by Japan Institute of Invention & Innovation (Kogi No. 2001-1745, published on Mar. 15, 2001), pp. 7 to 12.

The cellulose acylate may be used either singly or in combination of two or more thereof, if the substituent(s), degree of substitution, degree of polymerization, distribution of molecular weight, and the like are within the aforementioned ranges.

### [Additive to Cellulose Acylate]

To a cellulose acylate solution, in addition to a compound that lowers moisture permeability, any of various additives according to the usage (for example, a ultraviolet absorber, a plasticizer, a deterioration preventing agent, fine particles, and an optical-characteristic controlling agent) may be added, in any preparation step. As to the timing at which the additive(s) is added, the additive(s) may be added in any of the dope production steps. The additive(s) may be added in the last step of the dope preparation steps, to control the properties of the resultant composition.

The additive(s) may be in a solid or oily state. That is, there is no particular limitation to the melting points or boiling points of the additives. For example, a ultraviolet absorber having a melting point of 20° C. or less and a ultraviolet absorber having a melting point of 20° C. or more are used in combination; or, similarly, plasticizers may be used in combination. These measures are described, for example, in JP-A-2001-151901.

### (Ultraviolet Absorber)

Any kind of ultraviolet absorber can be selected according to the purpose of use, and examples of the UV absorber that can be used include those of salicylate ester-series, benzophenone-series, benzotriazole-series, benzoate-series, cyanoacrylate-series, and nickel complex-series; and a benzophenone-series, benzotriazole-series, or salicylate ester-series UV absorber is preferable.

Examples of the benzophenone-series ultraviolet absorber include 2,4-dihydroxybenzophenone, 2-hydroxy-4-acetoxybenzophenone, 2-hydroxy-4-methoxybenzophenone, 2,2′-di-hydroxy-4-methoxybenzophenone, 2,2′-di-hydroxy-4,4′-methoxybenzophenone, 2-hydroxy-4-n-octoxybenzophenone, 2-hydroxy-4-dodecyloxybenzophenone, and 2-hydroxy-4-(2-hydroxy-3-methacryloxy)propoxybenzophenone.

Examples of the benzotriazole-series ultraviolet absorber include 2-(2′-hydroxy-3′-tert-butyl-5′-methylphenyl)-5-chlorobenzotriazole, 2-(2′-hydroxy-5′-tert-butylphenyl)benzotriazole, 2-(2′-hydroxy-3′,5′-di-tert-amylphenyl)benzotriazole, 2-(2′-hydroxy-3′,5′-di-tert-butylphenyl)-5-chlorobenzotriazole, and 2-(2′-hydroxy-5′-tert-octylphenyl)benzotriazole.

Examples of the salicylate ester-series ultraviolet absorber include phenyl salicylate, p-octylphenyl salicylate, and p-tert-butylphenyl salicylate.

Of the ultraviolet absorbers as enumerated in the above, in particular, 2-hydroxy-4-methoxybenzophenone, 2,2′-di-hydroxy-4,4′-methoxybenzophenone, 2-(2′-hydroxy-3′-tert-butyl-5′-methylphenyl)-5-chlorobenzotriazole, 2-(2′-hydroxy-5′-tert-butylphenyl)benzotriazole, 2-(2′-hydroxy-3′,5′-di-tert-amylphenyl)benzotriazole, 2-(2′-hydroxy-3′,5′-di-tert-butylphenyl)-5-chlorobenzotriazole are particularly preferable.

It is preferable to use two or more ultraviolet absorbers having different absorption wavelength in combination, because great shielding ability can be obtained in a wide wavelength range. As the ultraviolet absorber for liquid crystal, preferable one is a ultraviolet absorber which is excellent in absorption ability for ultraviolet ray of wavelength 370 nm or lower, from the viewpoint of prevention of degradation of the liquid crystal, and which has less absorption of visible light of wavelength 400 nm or higher, from the viewpoint of displaying ability of the liquid crystal. Examples of the particularly preferable ultraviolet absorber include the aforementioned benzotriazole-series compounds, benzophenone-series compounds, and salicylate ester-series compounds. Among these, benzotriazole-series compounds are especially preferable, because of little coloration which is unnecessary against cellulose ester.

Further, as the UV absorber, use can also be made of any of the compounds described in JP-A-60-235852, JP-A-3-199201, JP-A-5-1907073, JP-A-5-194789, JP-A-5-271471, JP-A-6-107854, JP-A-6-118233, JP-A-6-148430, JP-A-7-11056, JP-A-7-11055, JP-A-7-11056, JP-A-8-29619, JP-A-8-239509, and JP-A-2000-204173.

The amount of the ultraviolet absorber to be added is preferably 0.001 to 5 mass %, more preferably 0.01 to 1 mass %, to the cellulose acylate. When the amount to be added is not less than 0.001 mass %, the addition effect can be sufficiently exhibited, which is preferable, and when the amount to be added is not more than 5 mass %, the ultraviolet absorber can be prohibited from being bleed out on the film surface, which is preferable.

Further, the ultraviolet absorber may be added at the same time upon dissolving a cellulose acylate, or may be added into the cellulose acylate solution (dope) after dissolution. It is particularly preferred that an ultraviolet absorber solution is added to the dope immediately before casting, by means of a static mixer or the like, thereby optical absorption characteristics can be easily controlled.

### (Deterioration Preventing Agent)

The deterioration preventing agent can prevent cellulose triacetate etc. from its degradation and decomposition. Examples of the deterioration preventing agent include butyl amine, hindered amine compounds (JP-A-8-325537), guanidine compounds (JP-A-5-271471), benzotriazole-series UV absorbers (JP-A-6-235819), and benzophenone-series UV absorbers (JP-A-6-118233).

### (Plasticizer)

The plasticizer that can be used in the present invention is preferably a phosphoric acid ester or a carboxylic acid ester. Examples of the phosphate-series plasticizer include triphenyl phosphate (TPP), tricresyl phosphate (TCP), cresyl diphenyl phosphate, octyl diphenyl phosphate, biphenyl diphenyl phosphate (BDP), trioctyl phosphate, and tributyl phosphate. Examples of the carboxylate-series plasticizer include dimethyl phthalate (DMP), diethyl phthalate (DEP), dibutyl phthalate (DBP), dioctyl phthalate (DOP), diphenyl phthalate (DPP), diethyl hexyl phthalate (DEHP), triethyl o-acetylcitrate (OACTE), tributyl o-acetylcitrate (OACTB), triethyl acetyl citrate, tributyl acetyl citrate, butyl oleate, methyl acetyl ricinoleate, dibutyl sebacate, triacetin, tributyrin, butyl-phthalyl-butyl glycolate, ethyl phthalyl ethyl glycolate, methyl phthalyl ethyl glycolate, butyl-phthalyl-butyl glycolate. The plasticizer is more preferably selected from the aforementioned exemplified plasticizers. Further, the plasticizer is preferably a (di)pentaerythritol ester, a glycerol ester, or a diglycerol ester.

### (Peeling Accelerator)

Examples of the peeling accelerator include ethyl esters of citric acid.

### (Infrared Absorber)

Examples of the infrared absorber include those described in JP-A-2001-194522.

### (Dye)

Further, in the present invention, a dye may be added, to adjust the hue of the resultant film. The amount to be added of the dye is preferably 10 to 1,000 ppm, more preferably 50 to 500 ppm, in terms of ratio by mass to the cellulose acylate. The light piping of the cellulose acylate film can be reduced and the yellowish feel of the cellulose acylate film can be improved, by adding the dye(s) in this manner. The dye may be added together with a cellulose acylate and a solvent, when the cellulose acylate solution is prepared; or alternatively the dye may be added during or after the preparation of the solution. Further, the dye may be added in the ultraviolet absorber solution, which is to be in-line-added. The dyes described in JP-A-5-34858 may also be used.

### (Matting Agent Fine-Particles)

To the cellulose acetate film of the present invention, fine particles as a matting agent are preferably added. Examples of the fine particles that can be used in the present invention include silicon dioxide, titanium dioxide, aluminum oxide, zirconium oxide, calcium carbonate, talc, clay, calcined kaolin, calcined calcium silicate, hydrated calcium silicate, aluminum silicate, magnesium silicate, and calcium phosphate. The fine particles are preferably those containing silicon, from the viewpoint of obtaining low turbidity, and particularly silicon dioxide is preferable. Fine particles of silicon dioxide are preferably those having a primary average particle diameter of 20 nm or less and an apparent specific gravity of 70 g/L or more. Particles having a primary average particle diameter as small as 5 to 16 nm are able to reduce the haze of the film, and are hence more preferable. The apparent specific gravity is preferably 90 to 200 g/L or more, and more preferably 100 to 200 g/L or more. A larger apparent specific gravity makes it possible to prepare a high concentration dispersion, to thereby better haze and coagulation, which is preferable.

The fine particles generally form secondary particles having an average particle diameter of 0.1 to 3.0 μm; and the fine particles exist in the form of a coagulate of primary particles in the film, to thereby being capable of forming irregularities 0.1 to 3.0 μm in size on the surface of the film. The secondary average particle diameter is preferably 0.2 μm or more and 1.5 μm or less, more preferably 0.4 μm or more and 1.2 μm or less, and most preferably 0.6 μm or more and 1.1 μm or less. Herein, the primary particle diameter and the secondary particle diameter are determined in the following manner: Particles in the film are observed by a scanning type electron microscope to measure the diameter of a circumscribed circle of a particle as a particle diameter. Further, 200 particles each in a different site or place are observed, to calculate an average of the diameters of these particles to determine an average particle diameter.

As the fine particles of silicon dioxide, for example, commercially available products under such trade names as Aerosil R972, R972V, R974, R812, 200, 200V, 300, R202, OX50, TT600 (trade names, manufactured by Nippon Aerosil Co., Ltd.) may be used. The fine particles of zirconium oxide are commercially available, for example, under such trade names as Aerosil R976 and R811 (trade names, manufactured by Nippon Aerosil Co., Ltd.), which may be used in the present invention.

Of those fine particles, Aerosil 200V and Aerosil R972V are particularly preferable, since they are fine particles of silicon dioxide having an average primary particle diameter of 20 nm or less and an apparent specific gravity of 70 g/L or more, and having a large effect of dropping friction coefficient, while maintaining the low turbidity of a resulting optical film.

To obtain a cellulose acylate film containing particles having a small secondary average particle diameter, several methods can be applied in the process of preparing a dispersion of fine particles. For example, in one method, a fine-particle dispersion obtained by mixing and stirring a solvent and fine particles, is prepared in advance. This fine particle dispersion is added into a small amount of a cellulose acylate solution which is separately prepared, and the mixture is dissolved with stirring. Then, the obtained mixture is further mixed in a main cellulose acylate dope solution. This method is a preferable preparation method in the point that the silicon dioxide fine-particles are well dispersed and are scarcely re-coagulated. Besides the above method, there is a method in which a small amount of a cellulose ester is added to a solvent, dissolved with stirring, fine particles are added thereto, and followed by dispersing by a dispersing apparatus, to obtain a fine-particle addition solution, which is sufficiently mixed with a dope solution by using an inline mixer. The present invention is not particularly limited by those methods, but the concentration of silicon dioxide when silicon dioxide fine-particles are mixed with and dispersed in, for example, a solvent is preferably 5 to 30 mass %, more preferably 10 to 25 mass %, and most preferably 15 to 20 mass %. The higher the concentration of the dispersion is, the lower the liquid turbidity in relation to the amount to be added is and the more greatly the haze and coagulate are bettered, and thus a higher concentration of silicon dioxide is preferable. The amount of the matting agent to be added in the final dope solution of the cellulose acylate is preferably 0.01 to 1.0 g/m², more preferably 0.03 to 0.3 g/m², and most preferably 0.08 to 0.16 g/m².

Preferable examples of a lower alcohol to be used as the solvent include methyl alcohol, ethyl alcohol, propyl alcohol, isopropyl alcohol, and butyl alcohol. As the solvent other than the lower alcohol, a solvent which is usually used for forming a cellulose ester film is preferably used, though not particularly limited to these solvents.

### [Ratios of Compounds to be Added]

In the cellulose acylate film of the present invention, the total amount of compounds having a molecular weight of 3,000 or less is preferably 5 to 45%, more preferably 10 to 40%, and further preferably 15 to 30%, to the mass of the cellulose acylate. These compounds include, as mentioned above, compounds lowering optical anisotropy, agents for controlling wavelength dispersion, ultraviolet absorbers (preventives), plasticizers, deterioration preventing agents, fine particles, releasing agents (peeling accelerators), and infrared absorbers. The molecular weight of each of these compounds is preferably 3,000 or less, more preferably 2,000 or less. If the total amount of these compounds is too small, the nature of cellulose acylate as a single substance may tend to be exhibited, resulting a problem, for example, that the optical performances or physical strength of the film tend to vary due to the change of temperature or humidity. On the other hand, if the total amount of these compounds is too large, the amount of the compounds may exceed the limit in which the compounds are compatible in the cellulose acylate film, and as a result, these compounds may tend to precipitate on the film surface to cause the film to be whitened or made whitely turbid (flowing or bleeding out from a film).

### [Organic Solvent of Cellulose Acylate Solution]

The cellulose acylate film is preferably prepared according to a solvent cast method. In the solvent cast method, a solution (dope) in which a cellulose acylate is dissolved in an organic solvent is used, to prepare a film. The organic solvent, which is preferably used as a main solvent, is preferably one selected from an ether having 3 to 12 carbon atoms, a ketone having 3 to 12 carbon atoms, an ester having 3 to 12 carbon atoms, and a halogenated hydrocarbon having 1 to 7 carbon atoms. The halogen in the halogenated hydrocarbon is preferably chlorine. The hydrogen atom in the halogenated hydrocarbon is substituted with a halogen in an amount of preferably 25 to 75 mol %, more preferably 30 to 70 mol %, further preferably 35 to 65 mol %, and most preferably 40 to 60 mol %. Dichloromethane is particularly preferable as a main solvent. The ester, ketone, or ether may have a cyclic structure. A compound having two or more functional groups of ester, ketone or ether (i.e. —O—, —CO—, or —COO—) can also be used as a main solvent. The organic solvent may have another functional group, such as an alcoholic hydroxyl group. If the main solvent is a compound having two or more functional groups, the number of carbon atoms can be within any of the above ranges defined for the compound having any of the functional groups. A mixture solution containing 75 to 89 parts by mass of a halogen-containing solvent and 11 to 25 parts by mass of an alcohol-series solvent, is most preferable. The alcohol is preferably monovalent. The hydrocarbon moiety of the alcohol may be straight-chain, branched or cyclic. The hydrocarbon moiety is preferably a saturated aliphatic hydrocarbon. Methanol is particularly preferable.

For a cellulose acylate film, a chlorine-containing halogenated hydrocarbon may be used as a main solvent, or a non-chlorine-containing solvent may be used as a main solvent, as described in “Kokai Giho” by Japan Institute of Invention and Innovation, 2001-1745 (pp. 12 to 16), but there is no particular limitation to these.

In addition, the following patent publications disclose the solvent, as well as including its dissolution method. Examples of the solvent and the like include those described in JP-A-2000-95876, JP-A-12-95877, JP-A-10-324774, JP-A-8-152514, JP-A-10-330538, JP-A-9-95538, JP-A-9-95557, JP-A-10-235664, JP-A-12-63534, JP-A-11-21379, JP-A-10-182853, JP-A-10-278056, JP-A-10-279702, JP-A-10-323853, JP-A-10-237186, JP-A-11-60807, JP-A-11-152342, JP-A-11-292988, JP-A-11-60752, and JP-A-11-60752. Further, those patent publications describe solution properties and co-existence materials that are made to coexist, and each publication may be used as a reference.

### [Producing Process of Cellulose Acylate Film]

### (Dissolution Step)

With regard to the preparation of the cellulose acylate solution (dope), there is no particular limitation to a method used to dissolve cellulose acylate. The dissolution may be carried out at the room temperature, or alternatively the dissolution may be carried out by a cooling dissolution method, a high-temperature dissolution method, or a combination of these methods. As to the preparation of the cellulose acylate solution, and the concentration and filtration of the solution associated with the dissolution step, the production processes described in detail in “Kokai Giho” by Japan Institute of Invention and Innovation Kogi No. 2001-1745, published on Mar. 15, 2001, pp. 22 to 25 are preferably used.

The dope transparency of the cellulose acylate solution is preferably 85% or more, more preferably 88% or more, and further preferably 90% or more. It can be thereby confirmed that various additives are sufficiently dissolved in the cellulose acylate dope solution. As to a specific method to calculate the dope transparency, the dope solution is injected into a glass cell which is 1 cm by 1 cm square, to measure the absorbance of the solution at a wavelength of 550 nm by using a spectrophotometer (UV-3150, trade name, manufactured by Shimadzu Corporation). The absorbance of the solvent may be measured as a control in advance, to calculate the transparency of the cellulose acylate solution from the ratio of the absorbance of the solution to that of the control.

### (Casting, Drying and Winding Steps)

Next, a method of producing a film by using the cellulose acylate solution will be explained. As a method and apparatus for producing the cellulose compound film of the present invention, use can be made of the solvent cast film-forming method and solvent cast film-forming apparatus conventionally employed in forming cellulose triacetate film. Those will be explained in detail hereinbelow, but the invention is not limited to those. A dope (a cellulose acylate solution) prepared in a dissolution machine (pot) is once stored in a storage pot, and, after defoaming to remove the foams in the dope, the dope is subjected to the final preparation. The dope is discharged from a dope exhaust and fed into a pressure die via, for example, a pressure constant-rate gear pump whereby the dope can be fed at a constant flow rate at a high accuracy depending on a rotational speed. From a pipe sleeve (slit) of the pressure die, the dope is uniformly cast onto a metallic support continuously running in the casting section. At the peeling point where the metallic support has almost rounded in one cycle, the half-dried dope film (also called a web) is peeled from the metallic support. The obtained web is clipped at both ends and dried by conveying with a tenter while maintaining the width at a constant level. Subsequently, the thus-obtained web film is mechanically conveyed with rolls in a dryer, to complete the drying, followed by winding with a winder into a rolled shape in a given length. Combination of the tenter and rolls in the dryer may vary depending on the purpose. In the solvent cast film-forming method utilized to produce a silver halide photographic light-sensitive material or a functional protective film that is an optical part for electronic displays, which are major application usages of cellulose acylate films, a coater is additionally employed in many cases, in addition to the solvent cast film-forming apparatus, so as to treat the film surface by providing, for example, an undercoat layer, an antistatic layer, an anti-halation layer or a protective layer. These production steps are described in detail in “Hatsumei Kyokai Kokai Giho” (Journal of Technical Disclosure) (Kogi No. 2001-1745, published Mar. 15, 2001, Japan Institute of Invention and Innovation), pp. 25 to 30, and they are classified into casting (including co-casting), metal supports, drying, releasing (peeling), etc., which can be preferably used in the present invention.

The thickness of the cellulose acylate film is preferably 10 to 120 μm, more preferably 20 to 100 μm, and further preferably 30 to 90 μm.

### (Stretching)

The cellulose acetate film is preferably subjected to stretching, to adjust the retardation. In particular, when the in-plane retardation value of a cellulose acetate film is to be made a high value, use may be made of a method of positively stretching said film in the transverse direction, for example, a method of stretching the produced cellulose acylate film, as described, for example, in JP-A-62-115035, JP-A-4-152125, JP-A-4-284211, JP-A-4-298310, and JP-A-11-48271.

Stretching of the film is carried out under the condition of the ordinary temperature or under heating. The temperature to be attained under the heating is preferably the same to or lower than the glass transition temperature of the film. The stretching of the film may be carried out by uniaxial stretching only in the longitudinal or transverse direction, or biaxial stretching in a simultaneous or successive manner. The stretching is generally in the range of from 1 to 200%, preferably in the range of from 1 to 100%, and particularly preferably in the range of from 1 to 50%.

In order to suppress light leakage when the thus-obtained film, e.g. a polarizing plate, is viewed from a slant direction, it is necessary to arrange the transmission axis of a polarizer in parallel to the in-plane slow-phase axis (retardation axis) of the cellulose acylate film. Generally, the transmission axis of a roll film-shaped polarizer which is continuously produced, is parallel to the transverse direction of the roll film. Thus, in order to apply the roll film-shaped polarizer continuously to a protective film composed of the roll film-shaped cellulose acylate film to make lamination of them, it is necessary that the in-plane slow-phase axis of the roll film-shaped protective film is parallel to the transverse direction of the film. Thus, it is preferable to stretch the cellulose acylate film much in the transverse direction. Further, the stretching may be carried out in the course of the film-forming step, or a roll of raw film formed and wound may be stretched. In the former case, the film may be stretched in the condition that the film contains a residual solvent. The film can be preferably stretched when the amount of the residual solvent is 2 to 30% by mass.

The film thickness of the cellulose acylate film obtained after drying may vary depending on the purpose of use, but it is generally in a range, preferably from 5 to 500 μm, more preferably 20 to 300 μm, and particularly preferably 30 to 150 μm. Further, the film thickness of the cellulose acylate film is preferably 40 to 110 μm, when the film is applied to optical devices, particularly VA liquid crystal display devices. In order to control the thickness of the film, it is sufficient to control, for example, the concentration of the solid contained in the dope, the slit gap of a die nozzle, the extrusion pressure from the die, and the speed of the metal support, to attain a target thickness.

The width of the cellulose acylate film obtained in the above manner is preferably 0.5 to 3 m, more preferably 0.6 to 2.5 m and further preferably 0.8 to 2.2 m. The film is wound in a length of preferably 100 to 10,000 m, more preferably 500 to 7,000 m, and further preferably 1,000 to 6,000 m, per roll. When the film is wound, at least one end of the roll is preferably knurled. The width of the knurl is preferably 3 mm to 50 mm, and more preferably 5 mm to 30 mm, and the height of the knurl is preferably 0.5 to 500 μm, and more preferably 1 to 200 μm. The film may be knurled on one side or both sides.

The dispersion (scattering) of a Re value in the transverse direction of the film is preferably ±5 nm, and more preferably ±3 nm. Also, the dispersion of a Rth value in the transverse direction is preferably ±10 nm, and more preferably ±5 nm. Further, each dispersion of Re value and Rth value in the longitudinal direction is preferably within the same range as to that of the dispersion in the transverse direction.

### [Optical Performances of Cellulose Acylate Film]

Herein, in the present invention, the Re(λ) and the Rth(λ) indicate the in-plane retardation and the retardation in the direction of the thickness, respectively, at the wavelength λ (nm). The Re(λ) can be measured by making light of wavelength λ nm incident in the direction of the normal of the film, in KOBRA 21ADH (trade name, manufactured by Oji Scientific Instruments). The Rth(λ) is a value that can be calculated by KOBRA 21ADH based on: (i) the retardation values measured in total three directions, these retardation values including the above Re(λ), the retardation value measured by allowing light of wavelength λ nm to be incident from a direction inclined at an angle of +40° with the direction of the normal of the film by adopting the slow phase axis (which is determined by the KOBRA 21ADH) in the plane as a slant axis (rotation axis), and the retardation value measured by allowing light of wavelength λ nm to be incident from a direction inclined at an angle of −40° with the direction of the normal of the film by adopting the slow phase axis in the plane as a slant axis (rotation axis); (ii) an hypothetical value of the average refractive index; and (iii) a film thickness input.

Herein, as the hypothetical value of the average refractive index, use may be made, for example, of values described in “Polymer Handbook” (JOHN WILEY & SONS, INC.) and values described in catalogues of various optical films. Unknown average refractive indexes may be determined by Abbe refractometer. Average refractive indexes of major optical films are exemplified in below: cellulose acylate (1.48), cycloolefin polymer (1.52), polycarbonate (1.59), polymethyl methacrylate (1.49), and polystyrene (1.59). KOBRA 21ADH can calculate nx, ny, and nz, by inputting these hypothetical values of the average refractive index and the film thickness.

It is preferable that the Re(λ) retardation value and the Rth(λ) retardation value satisfy the following mathematical expressions (2) and (3), respectively, to widen the angle of field of view of a liquid crystal display device, particularly a VA mode liquid crystal display device. Further, this is particularly preferable when the cellulose acylate film is used for the protective film on the liquid crystal cell side of the polarizing plate.

0 nm≦Re≦200 nm Mathematical expression (2)

0 nm≦Rth≦400 nm Mathematical expression (3)

In the above mathematical expressions, Re and Rth each are a value (unit: nm) measured at wavelength (λ) of 590 nm (in the present invention, Re and Rth each mean a value at this wavelength, unless otherwise specified).

When the cellulose compound file is used in a VA mode, there are two types of structures: a structure (two-film type) in which the film is applied to each side of a cell, i.e. the total two films are utilized; and a structure (one-film type) in which the film is applied only one side of a cell.

In the case of the two-film type, the Re is preferably 20 to 100 nm, more preferably 30 to 70 nm; and the Rth is preferably 70 to 300 nm, more preferably 100 to 200 nm.

In the case of the one-film type, the Re is preferably 30 to 150 nm, more preferably 40 to 100 nm; and the Rth is preferably 100 to 300 nm, more preferably 150 to 250 nm.

### [Moisture Permeability of Film]

The moisture permeability of a cellulose acylate film to be used for an optical compensation sheet is preferably 400 to 2,000 g/m²·24 h, more preferably 500 to 1,800 g/m²·24 h, and particularly preferably 600 to 1,600 g/m²·24 h, based on the case where the film thickness be 80 μm in measurement under the conditions of temperature 60° C. under humidity 95% RH (relative humidity) according to JIS Standard, JIS Z0208. If the moisture permeability is too large, the absolute values of the Re value and Rth value of the film, which values are humidity dependent, strongly tends to exceed 0.5 nm/% RH, and also the absolute value of the Re value and Rth value, which values are humidity dependent, strongly tends to exceed 0.5 nm/% RH in the case where an optical anisotropic layer is laminated on the cellulose acylate film to make an optical compensation film, each of which phenomena is unpreferable. When this optical compensation sheet or polarizing plate is assembled into a liquid crystal display device, it causes variation in color hue or deterioration in the angle of field of view. Further, if the moisture permeability of the cellulose acylate film is too small, an adhesive is inhibited from being dried due to the cellulose acylate film, causing inferior adhesion, when, for example, the cellulose acylate film is applied to each side of the polarizing film to make a polarizing plate.

If the film thickness of the cellulose acylate film is thick, the moisture permeability becomes small, whereas if the film thickness is thin, the moisture permeability becomes large. Thus, the moisture permeability of a sample of any film thickness must be converted, based on that of a standard sample of thickness 80 μm. The conversion of the film thickness is made according to the following equation: (Moisture permeability converted to thickness 80 μm)=(Measured moisture permeability)×(Measured film thickness μm/80 μm)

As a method of measuring the moisture permeability, the method described in “Properties of Polymers II” (Polymer Experiment Lesson 4, Kyoritsu Shuppan), pp. 285 to 294: Measurement of Amount of Vapor Transmission (Mass method, Temperature gauge method, Vapor pressure method, and Adsorption amount method), may be applied. That is, a 70 mmφ cellulose acylate film sample according to the present invention is humidity-controlled at 25° C. under humidity 90% RH and at 60° C. under humidity 95% RH, respectively for 24 hours, to measure the amount of water per unit area (g/m²), by using a moisture permeability tester (trade name: KK-709007, manufactured by Toyo Seiki Seisaku-sho, Ltd.), according to JIS Z-0208, and then the moisture permeability is calculated from the following equation: (Moisture permeability)=(Mass after moisture control)−(Mass before moisture control)

### [Amount of Residual Solvent in Film]

It is preferable to dry the cellulose acylate film in the condition that the amount of a residual solvent is decreased to an amount range from 0.01 to 1.5% by mass, more preferably 0.01 to 1.0% by mass, to the cellulose acylate film. When the amount of a residual solvent in a transparent support is decreased to 1.5% or less, curling can be suppressed or prevented. The amount of a residual solvent is more preferably 1.0% or less. This is assumed to be based on that the amount of a residual solvent may be reduced upon the film formation by the aforementioned solvent-casting method, leading to a reduced free volume, which would be a main factor of the effect.

### [Coefficient of Hygroscopic Swelling of Film]

The coefficient of hygroscopic swelling (expansion) of the cellulose acylate film is preferably 30×10⁻⁵/% RH or less, more preferably 15×10⁻⁵/% RH or less, and further preferably 10×10⁻⁵/% RH or less. Further, the coefficient of hygroscopic swelling is preferably as small as possible, but it is generally a value of 1.0×10⁻⁵/% RH or more. The coefficient of hygroscopic swelling indicates an amount of change in the length of a sample when relative humidity is changed under a fixed temperature condition. By controlling the coefficient of the hygroscopic swelling, the cellulose acylate film can be used as an optical compensation film support, while maintaining the optical compensation function of the optical compensation film, and with preventing an architrave-like (or frame-like) rise in transmission, i.e. light leakage due to strain.

### [Surface Treatment]

A cellulose acylate film may be subjected to a surface treatment, if necessary, in order to achieve enhanced adhesion between the cellulose acylate film and each functional layer (e.g., subbing or undercoat layer, and backing layer). For example, a glow discharge treatment, an ultraviolet ray treatment, a corona discharge treatment, a flame treatment, an acid treatment, and an alkali treatment may be applied. The glow discharge treatment referred to herein may be a treatment with low-temperature plasma (thermal plasma) generated in a low-pressure gas having a pressure of 10⁻³ to 20 Torr, or preferably with plasma under the atmospheric pressure. A plasma excitation gas is a gas which can be excited to plasma under conditions as described above, and examples thereof include argon, helium, neon, krypton, xenon, nitrogen, carbon dioxide, flons such as tetrafluoromethane, and a mixture thereof. Details thereof are described in “Hatsumei Kyokai Kokai Giho” (Kogi No. 2001-1745, published Mar. 15, 2001, Japan Institute of Invention and Innovation), pp. 30 to 32, which detailed techniques can be preferably used in the present invention.

### [Alkali Saponifying Treatment]

The alkali saponifying treatment is preferably conducted, by directly immersing the cellulose acylate film into a bath of a saponifying solution, or by applying a saponifying solution onto the cellulose acylate film. Examples of the application method include a dip coating method, a curtain coating method, an extrusion coating method, a bar coating method, and an E-type coating method. As the solvent in the alkali saponifying treatment coating solution, it is preferable to employ a solvent which has an excellent wettability appropriate for applying the saponifying solution to a cellulose acylate film, and which can hold a favorable surface state without forming any irregularity on the cellulose acylate film surface due to the saponifying solution solvent. More specifically speaking, it is preferable to use an alcoholic solvent, and isopropyl alcohol is particularly preferable therefor. It is also possible to employ an aqueous solution of a surfactant as the solvent. As the alkali in the alkali saponifying solution, it is preferable to use an alkali soluble in the above-described solvent, and KOH or/and NaOH is further preferable therefor. It is preferable that the saponifying (coating) solution has a pH value of 10 or more, more preferably 12 or more. Concerning the reaction conditions, it is preferable to perform the alkali saponification at room temperature for 1 second or longer but 5 minutes or shorter, more preferably for 5 seconds or longer but 5 minute or shorter, and particularly preferably for 20 seconds or longer but 3 minutes or shorter. After the completion of the alkali saponification reaction, it is preferable to wash with water; or wash with an acid and then wash with water, the face coated with the saponifying solution.

### [Functional Layers]

The cellulose acylate film of the present invention can be applied to optical articles or to photosensitive materials, as the usage applications. Particularly, it is preferred that the optical article is a liquid crystal display device. Further, it is more preferable that the liquid crystal display device has a configuration wherein a liquid crystal cell carrying a liquid crystal between two sheets of electrode substrates, two sheets of polarizers disposed at both sides of the liquid crystal cell one by one, and at least one optical compensating sheet disposed between the liquid crystal cell and the polarizer. As these liquid crystal display devices, TN, IPS, FLC, AFLC, OCB, STN, ECB, VA, and HAN are preferable.

When the cellulose acylate film in accordance with the present invention is used for the aforementioned optical articles, any of various kinds of functional layers may be provided on the film. Examples of the functional layer include an antistatic layer, a hardened resin layer (transparent hard coat layer), an anti-reflection layer, an enhanced-adhesion layer, an anti-glare layer, an optical compensating layer, an orientating layer, and a liquid crystal layer. As the functional layer and material therefor, which may be used in the cellulose acylate film of the present invention, a surface-active agent, a sliding agent, and a matting agent, an anti-static layer, and a hard-coat layer are enumerated, details of which are described in “Kokai Giho of Japan Institute of Invention & Innovation” (Kogi No. 2001-1745, published on Mar. 15, 2001), pp. 32 to 45, which can be preferably used.

### [Polarizing Plate]

The optical film is particularly useful for a polarizing plate protective film. When the film is used as a polarizing plate-protective film, the production method of polarizing plate is not particularly limited, but the polarizing plate may be produced in a usual manner. For example, there is a method of producing a polarizing plate, comprising the steps of: alkali-treating the obtained cellulose acylate film; and sticking, with using an aqueous solution of completely saponificated polyvinyl alcohol, the alkali-treated film one by one onto each side of a polarizer produced by dipping a polyvinyl alcohol film in an iodine solution, followed by stretching. In place of the alkali treatment, an enhanced adhesion processing, as described in JP-A-6-94915 and JP-A-6-118232, may be adopted to the aforementioned production method.

Examples of the adhesive that can be used in adhering the treated side of the protective film and the polarizer, include polyvinyl alcohol-series adhesives, such as polyvinyl alcohol and polyvinyl butyral; and vinyl-series latexes, such as butyl acrylate.

A polarizing plate is generally composed of a polarizer and protecting films to protect both surfaces of the polarizer, and the thus-prepared polarizing plate is further provided with a protect film stuck to one surface of the polarizing plate, and a separation film stuck to the opposite surface of the polarizing plate. The protect film and the separation film are used, in order to protect the polarizing plate when the polarizing plate is shipped and subjected to a product testing or the like. In this case, the protect film is stuck in order to protect the surface of a polarizing plate, and the film is used at the side of the surface opposite to the surface with which the polarizing plate is stuck to a liquid crystal plate. Meanwhile, the separation film is used to cover an adhesive layer to be stuck to the liquid crystal plate, and the film is used at the same side as the surface with which the polarizing plate is stuck to a liquid crystal plate.

In a liquid crystal display device, usually, a substrate containing liquid crystals is disposed between two polarizing plates. A polarizing-plate protective film to which the optical film of the present invention is applied can exhibit excellent display performances, regardless of the site the film is to be disposed. In particular, because a transparent hard coat layer, an anti-glare layer, an anti-reflection layer, and the like layers are disposed to a polarizing-plate protective film to be disposed at the outermost surface at the displaying side of a liquid crystal display device, employment of the aforementioned polarizing-plate protective film of the present invention at this site is especially preferable.

### [Optical Compensation Film]

The cellulose acylate film of the present invention can be utilized in various usage applications. It is especially effective when the cellulose acylate film is used as an optical compensation film for liquid crystal display device. Herein, the optical compensation film means an optical material that is generally used in a liquid crystal display device to compensate a phase difference, and that has the same meaning, for example, as a phase difference plate and an optical compensation sheet. The optical compensation film has birefringence characteristics, and can be used for the purpose of eliminating coloring on the displaying plane of a liquid crystal display device or improving the characteristics of the angle of field of view.

### (Constitution of Liquid Crystal Display Device)

When the cellulose acylate film itself is used as an optical compensation sheet, the transmission axis of a polarizing element (polarizer) and the slow phase axis of an optical compensation sheet composed of the cellulose acylate film can be arranged at any angle. A liquid crystal display device has, for example, a constitution of a liquid crystal cell carrying liquid crystal between two pieces of electrode substrates, two polarizing elements disposed on both surfaces of the liquid crystal cell, and at least one optical compensation sheet disposed between the liquid crystal cell and the polarizing element.

The liquid crystal layer of the liquid crystal cell can be formed, for example, by sealing a liquid crystal, in the space made by putting a spacer sandwiched between two pieces of substrates. The transparent electrode layer can be formed, for example, on the substrate as a transparent film containing an electric conductive substance. Further, to the liquid crystal cell, for example, a gas barrier layer, a hard coat layer, or an undercoat (or subbing) layer (used for adhesion of the transparent electrode layer) may be further provided. The aforementioned layers can be provided on the substrate. It is preferable that the thickness of the substrate for the liquid crystal cell is generally from 50 μm to 2 mm.

### (Kinds of Liquid Crystal Display Devices)

The cellulose acylate film of the present invention can be applied to liquid crystal cells of various display modes. As for the display modes, proposed are various modes, for example, TN (Twisted Nematic), IPS (In-Plane Switching), FLC (Ferroelectric Liquid Crystal), AFLC (Anti-ferroelectric Liquid Crystal), OCB (Optically Compensatory Bend), STN (Supper Twisted Nematic), VA (Vertically Aligned), ECB (Electrically Controlled Birefringence), and HAN (Hybrid Aligned Nematic). In addition, there are proposals of display modes that are obtained by orientation dividing of the aforementioned display modes. The cellulose acylate film of the present invention is effective in liquid crystal display devices with any display mode. Further, the cellulose acylate film is effective in any of transparent-type, reflection-type, and semitransparent-type liquid crystal display devices.

### (TN-Type Liquid Crystal Display Device)

The Cellulose Acylate Film of the Present Invention can be Used as a Support for an Optical compensation sheet that is used in TN type liquid crystal display devices having the liquid crystal cell of TN mode. The TN mode liquid crystal cell and the TN-type liquid crystal display device per se are well known for a long time. The optical compensation sheet that is used in TN-type liquid crystal display devices is described in, for example, JP-A-3-9325, JP-A-6-148429, JP-A-8-50206, and JP-A-9-26572, and also described in, for example, papers authored by Mori, et al. (Jpn. J. Appl. Phys., Vol. 36 (1997), p. 143, and Jpn. J. Appl. Phys., Vol. 36 (1997), p. 1068).

### (STN-Type Liquid Crystal Display Device)

The cellulose acylate film of the present invention may be used as a support for an optical compensation sheet that is employed in STN-type liquid crystal display devices installing a STN mode liquid crystal cell. In STN-type liquid crystal display devices, generally, cylindrical shape mesomorphism molecules in the liquid crystal cell is twisted in the range of 90 to 360 degrees, and the product (Δnd) of the refractive index anisotropy (Δn) and cell gap (d) of the cylindrical shape mesomorphism molecular is in the range of 300 to 1,500 nm. Regarding optical compensation sheets used for the STN type liquid crystal display devices, JP-A-2000-105316 describes in detail.

### (VA-Type Liquid Crystal Display Device)

The cellulose acylate film of the present invention can be particularly advantageously used as a support for an optical compensation sheet that is used in the VA-type liquid crystal display devices installing a VA mode liquid crystal cell. It is preferred that the Re retardation value is controlled to the range of from 0 to 150 nm and the Rth retardation value is controlled to the range of from 70 to 400 nm, respectively, for the optical compensation sheet that is used in the VA-type liquid crystal display device. It is more preferable that the Re retardation value is in the range of from 20 to 70 nm. In an embodiment where two sheets of optically anisotropic polymer films are used in a VA-type liquid crystal display device, it is preferred that the Rth retardation value of the film is in the range of from 70 to 250 nm. In an embodiment where one sheet of an optically anisotropic polymer film is used in a VA-type liquid crystal display device, it is preferred that the Rth retardation value of the film is in the range of from 150 to 400 nm. The VA-type liquid crystal display device may have an orientation dividing system, as described in, for example, JP-A-10-123576.

### (IPS-Type Liquid Crystal Display Device and ECB-Type Liquid Crystal Display Device)

The cellulose acylate film may also be particularly advantageously used as the support for the optical compensation sheet or as the protective film of the polarizing plate, in an IPS-type liquid crystal display device or ECB-type liquid crystal display device in which an IPS-mode or ECB-mode liquid crystal cell is assembled. In these modes, a mesomorphism (liquid crystal) material is oriented almost in parallel when a black color is displayed, and a mesomorphism molecule is oriented in parallel to the surface of the substrate in the condition that no voltage is applied, to display a black color. In these modes, the polarizing plate using the cellulose acylate film of the present invention contributes to improvement in color hue, expansion of the angle of field of view, and improvement in contrast. In these modes, it is preferable that use is made of, for at least one side of the two polarizing plates, a polarizing plate in which the cellulose acylate film of the present invention is used for the protective film (a cell-side protective film) disposed between the liquid crystal cell and the polarizing plate, of the protective films of the above polarizing plates on the upper and lower sides of the liquid crystal cell. It is more preferable that an optical anisotropic layer be disposed between the protective film of the polarizing plate and the liquid crystal cell, and that the retardation value of the disposed optical anisotropic layer be set to a value not more than twice the value of Δn·d of the liquid crystal layer.

### (OCB-Type Liquid Crystal Display Device and HAN-Type Liquid Crystal Display Device)

The cellulose acylate film of the present invention can also be advantageously used as a support for an optical compensation sheet that is used in an OCB-type liquid crystal display device having a liquid crystal cell of OCB mode, or used in a HAN-type liquid crystal display device having a liquid crystal cell of HAN mode. It is preferable that, in the optical compensation sheet used for an OCB-type liquid crystal display device or a HAN-type liquid crystal display device, the direction where the magnitude or absolute value of retardation becomes the minimum value exists neither in the optical compensation sheet plane nor in its normal direction. Optical properties of the optical compensation sheet for use in the OCB type liquid crystal display device or the HAN type liquid crystal display device are also determined by the optical properties of the optical anisotropy layer, by the optical properties of the support, and by the arrangement of the optical anisotropy layer and the support. JP-A-9-197397 describes, regarding the optical compensation sheet for use in the OCB type liquid crystal display device or HAN type liquid crystal display device. In addition, a paper by Mori et al. (Jpn. J. Appl. Phys., Vol. 38 (1999), p. 2837) describes about it.

### (Reflection-Type Liquid Crystal Display Device)

The cellulose acylate film of the present invention can also be advantageously used as an optical compensation sheet for the reflection-type liquid crystal display devices of TN-type, STN-type, HAN-type, or GH (Guest-host)-type. These display modes are well known for a long time. The TN-type reflection-type liquid crystal display devices are described in, for example, JP-A-10-123478, WO 98/48320, and Japanese Patent No: 3022477. The optical compensation sheet for use in a reflection type liquid crystal display device is described in, for example, WO 00/65384.

### (Other Liquid Crystal Display Devices)

The cellulose acylate film of the present invention can also be advantageously used as a support for an optical compensation sheet for use in ASM (Axially Symmetric Aligned Microcell) type liquid crystal display devices having a liquid crystal cell of ASM mode. The liquid crystal cell of ASM mode is characterized in that a resin spacer adjustable with its position maintains the thickness of the cell. Other properties of the liquid crystal cell of ASM mode are similar to the properties of the liquid crystal cell of TN mode. Regarding liquid crystal cells of ASM mode and ASM type liquid crystal display devices, descriptions can be found in a paper of Kume et al. (Kume et al., SID 98 Digest 1089 (1998)).

### [Hardcoat Film, Antiglare Film, and Antireflection Film]

The cellulose acylate film may also be preferably applied to a hardcoat film, an antiglare film, and an antireflection film. Any or all of the hardcoat layer, antiglare layer and antireflection layer may be provided on one or both surfaces of the cellulose acylate film of the present invention, for the purposes of improving the visibility of the flat panel displays of LCDs, PDPs, CRTs, ELs, and the like. Preferable embodiments of the antiglare film or antireflection film are described in detail in Japan Institute of Invention and Innovation, “Kokai Giho” Kogi No. 2001-1745, published on Mar. 15, 2001, pp. 54 to 57. The cellulose acylate film of the present invention can be preferably used in these embodiments.

### [Photographic Film Support]

The cellulose acylate film may be applied as a support of a silver halide photographic photosensitive material. Various raw materials, formulations, and process or treating methods, as described in the patent references referred to herein, may be applied. As regards the techniques, there are the detailed descriptions concerning color negatives in JP-A-2000-105445, and the cellulose acylate film of the present invention can be preferably used in the aforementioned color negatives. Further, the cellulose acylate film is also preferably applied as the support of a color reversal silver halide photographic photosensitive material, and various raw materials, formulations, and processing or treating methods, as described in JP-A-11-282119, may be applied thereto.

### [Transparent Substrate]

Since the cellulose acylate film is close to zero in the optical anisotropy and has excellent transparency, the cellulose acylate film may be used in place of a liquid crystal cell glass substrate of a liquid crystal display device, i.e. it may be used as a transparent substrate that seals a driving liquid crystal.

Because it is necessary that the transparent substrate that seals a liquid crystal be excellent in gas barrier properties, the cellulose acylate film of the present invention may be provided with a gas barrier layer on the surface thereof. There is no particular limitation on the shape and material of the gas barrier layer. Specifically, any of the following methods may be given, in which, on at least one of the surfaces of the cellulose acylate film of the present invention, SiO₂ or the like is vapor-deposited, or a coating layer of a polymer, such as a vinylidene chloride-series polymer or vinyl alcohol-series polymer, which is relatively high in gas barrier properties, is formed. Any of these methods may be appropriately used in the present invention.

Further, when the cellulose acylate film is used as a transparent substrate that seals a liquid crystal, it may be provided with a transparent electrode(s) to drive the liquid crystal by applying voltage. There is no particular limitation on the transparent electrode, but a metal film, metal oxide film or the like may be laminated, to thereby form the transparent electrode(s), on at least one of the surfaces of the cellulose acylate film. Of those, a film of a metal oxide is preferable, from the viewpoints of transparency, electrical conductivity, and mechanical characteristics. In particular, a thin film of indium oxide containing tin oxide primarily and 2 to 15% of zinc oxide, can be preferably used. The details of these techniques are disclosed in, for example, JP-A-2001-125079 and JP-A-2000-227603.

In order to control the Re value and Rth value of the cellulose compound film to be each within the preferable ranges, it is preferable to properly control the type and amount to be added of the compound represented by formula (1) or (2) to be contained (hereinafter, which may also be referred to as a retardation controlling agent), as well as a stretching ratio of the film. Specifically, in the cellulose compound film of the present invention, a suitable retardation controlling agent can be selected, to thereby control the Rth value in the target range, and the amount of the retardation controlling agent to be added and the ratio of stretching the film each can be properly set, to thereby control the Re value in the target range; and thus a cellulose compound film having a desired Re value and Rth value and a combination of these values can be obtained.

The cellulose compound composition of the present invention has good solubility in a halogen-containing mixture solution, and is useful to form a film which has a high Re value and a low Rth value, which attains high optical anisotropy, and which is excellent in optical performances. Thus, a cellulose derivative film obtained from this cellulose compound composition is excellent in optical performances.

The cellulose derivative film of the present invention can be preferably used as an optical film for a liquid crystal display device, and particularly as an optical compensation sheet.

### EXAMPLES

The present invention will be described in more detail based on the following examples. The materials, the amounts to be used, the proportions, the contents and procedures of treatment or processing, which will be shown in the examples, may be appropriately changed or modified, without departing from the spirit of the present invention. Therefore, the following examples are not interpreted as limiting of the scope of the present invention.

### Example 1

### Production of Exemplified Compound (11)

The Exemplified compound (11) was produced, according to the following scheme.

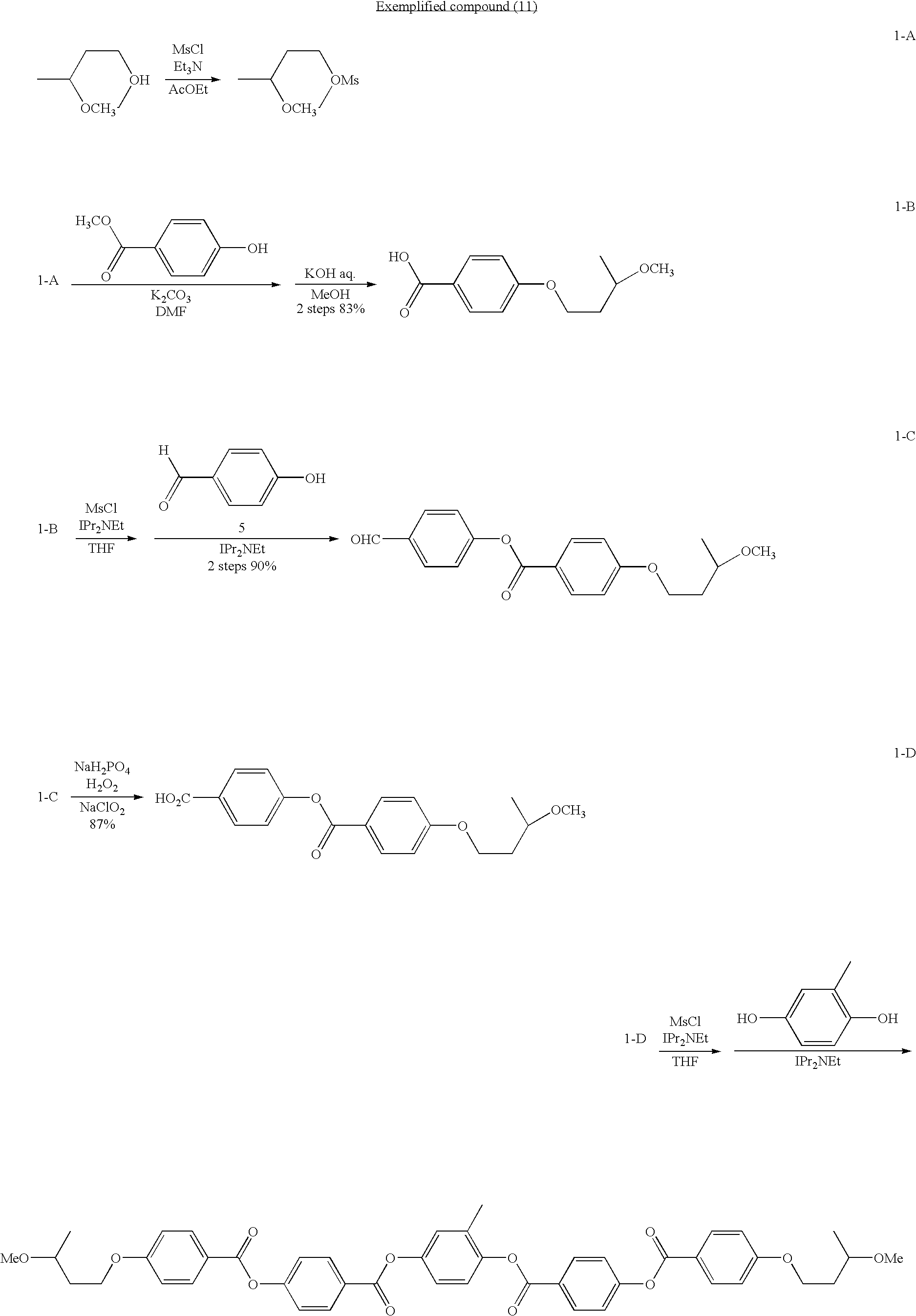

### [Production of Intermediate (1-A)]

To an ethyl acetate (AcOEt) solution containing 200 g (1.92 mol) of 3-methoxy-1-butanol, added was 185 g (1.83 mol) of triethylamine (Et₃N). After the reaction system was cooled to 2° C., 209 g (1.83 mol) of methanesulfonic acid chloride (MsCl) was added dropwise to the reaction system, while the temperature of the reaction system was kept at 15° C. or lower. After the dropwise addition was completed, the reaction system was stirred at 10° C. or lower for 30 minutes, and then the temperature of the reaction system was raised to the room temperature, followed by stirring for 3 hours. Water was added thereto, to separate the reaction solution, and the separated organic phase was washed with water, 1-N aqueous hydrochloric acid, and water, in this order. The resulting organic phase was dried over magnesium sulfate, and the solvent was distilled off under reduced pressure, to obtain 292.2 g of a crude intermediate (1-A) (yield 88%).

### [Production of Intermediate (1-B)]

To 1,600 ml of an N,N-dimethylformamide (DMF) solution containing 60 g (0.394 mol) of methyl parahydroxybenzoate, added was 70.8 g (0.512 mol) of potassium carbonate, and to the resultant mixture, added was 89 g (0.488 mol) of the intermediate (1-A). The temperature of the reaction system was raised to 100° C., followed by stirring for 6 hours. After the reaction was finished, the system was cooled. To the thus-cooled system, water and 1 L of ethyl acetate were added, to separate the resultant solution. The separated organic phase was washed with water, 1-N aqueous hydrochloric acid, and water, in this order. The organic phase was dried over magnesium sulfate, and the solvents were distilled off under reduced pressure. To the resultant crude product, 85 ml of methanol (MeOH) was added, and 240 ml of an aqueous solution containing 66.4 g (1.182 mol) of potassium hydroxide was added dropwise to the mixture. The reaction system was heated to 50° C., followed by stirring for 4 hours at that temperature. The completion of the reaction was confirmed by thin layer chromatography (TLC), and the reaction system was cooled to 5° C. The reaction solution was slowly added dropwise to 1 L of 1-N aqueous hydrochloric acid cooled to 5° C. The thus-produced crystals were collected by filtration, and dried, to obtain 73.4 g of an intermediate (1-B) (yield 83%).

### [Production of Intermediate (1-C)]

To 500 ml of a tetrahydrofuran solution containing 50 g (0.223 mol) of the intermediate (1-B), added was 17.3 ml (0.224 mol) of methansulfonic acid chloride, under ice-cooling, and to the resultant mixture, added slowly, dropwise, was 40 ml (0.230 mol) of N,N-diisopropylethylamine (IPr₂NEt). After the resultant mixture was stirred for one hour, 100 ml of a tetrahydrofuran solution containing 30.2 g (0.247 mol) of parahydroxybenzaldehyde, and 40 ml (0.230 mol) of N,N-diisopropylethylamine were added dropwise to the mixture. Then, to the mixture, 100 ml of a tetrahydrofuran solution containing 0.2 g of N,N-dimethylaminopyridine was added dropwise, followed by stirring under ice-cooling for one hour, then raising to the room temperature, and further stirring for 6 hours. To the reaction solution, water was added, to separate the solution, and the separated organic phase was washed with water, 1-N aqueous hydrochloric acid, and water, in this order. The resultant organic phase was dried over magnesium sulfate, and the solvent was distilled off under reduced pressure, to obtain 66 g of a crude intermediate (1-C) (yield 90%).

### [Production of Intermediate (1-D)]

Two hundred milliliters of an acetonitrile solution containing 43 g (0.13 mol) of the intermediate (1-C) was cooled to 10° C., and thereto 25 ml of an aqueous solution containing 4.21 g (0.027 mol) of sodium dihydrogenphosphate was added dropwise, and then 23 ml (0.195 mol) of a 30% aqueous hydrogen peroxide was added dropwise. To the mixture, 125 ml of an aqueous solution containing 15.9 g of sodium chlorite was slowly added dropwise. The temperature of the reaction system was raised to 50° C., followed by stirring for 3 hours. The completion of the reaction was confirmed by TLC, and the reaction system was cooled to 10° C. The reaction solution was slowly added dropwise to 1.5 L of 1-N aqueous hydrochloric acid cooled to 5° C. The thus-produced crystals were collected by filtration, and dried, to obtain 38.8 g of an intermediate (1-D) (yield 87%).

### [Production of Exemplified Compound (11)]

To 80 ml of a tetrahydrofuran solution containing 6 g (17 mmol) of the intermediate (1-D), added was 1.3 ml (17 mmol) of methansulfonic acid chloride, under ice-cooling, and to the resultant mixture, added slowly, dropwise, was 3.0 ml (17 mmol) of N,N-diisopropylethylamine. After the resultant mixture was stirred for one hour, to the mixture, 6.0 ml (34 mmol) of N,N-diisopropylethylamine was added, and then 30 ml of a tetrahydrofuran solution containing 1.0 g of methylhydroquinone was added dropwise. Then, to the mixture, 5 ml of a tetrahydrofuran (THF) solution containing 0.1 g of N,N-dimethylaminopyridine was added dropwise, followed by stirring under ice-cooling for one hour, then raising to the room temperature, and further stirring for 6 hours. The reaction solution was added dropwise to 1 L of methanol, and the thus-produced crystals were collected by filtration, and dried, to obtain 4.2 g of the Exemplified compound (11) (yield 69%).

The compound was identified by ¹H-NMR.

¹H-NMR (CDCl₃): δ 1.25 (d, 6H), 2.00 (m, 4H), 2.25 (s, 3H), 3.35 (s, 6H), 3.60 (m, 2H), 4.15 (m, 4H), 7.00 (d, 4H), 7.15 (m, 3H), 7.40 (m, 4H), 8.15 (m, 4H), 8.30 (m, 4H)

### Example 2

### Production of Exemplified Compound (1)

The Exemplified compound (1) was produced, according to the following scheme.

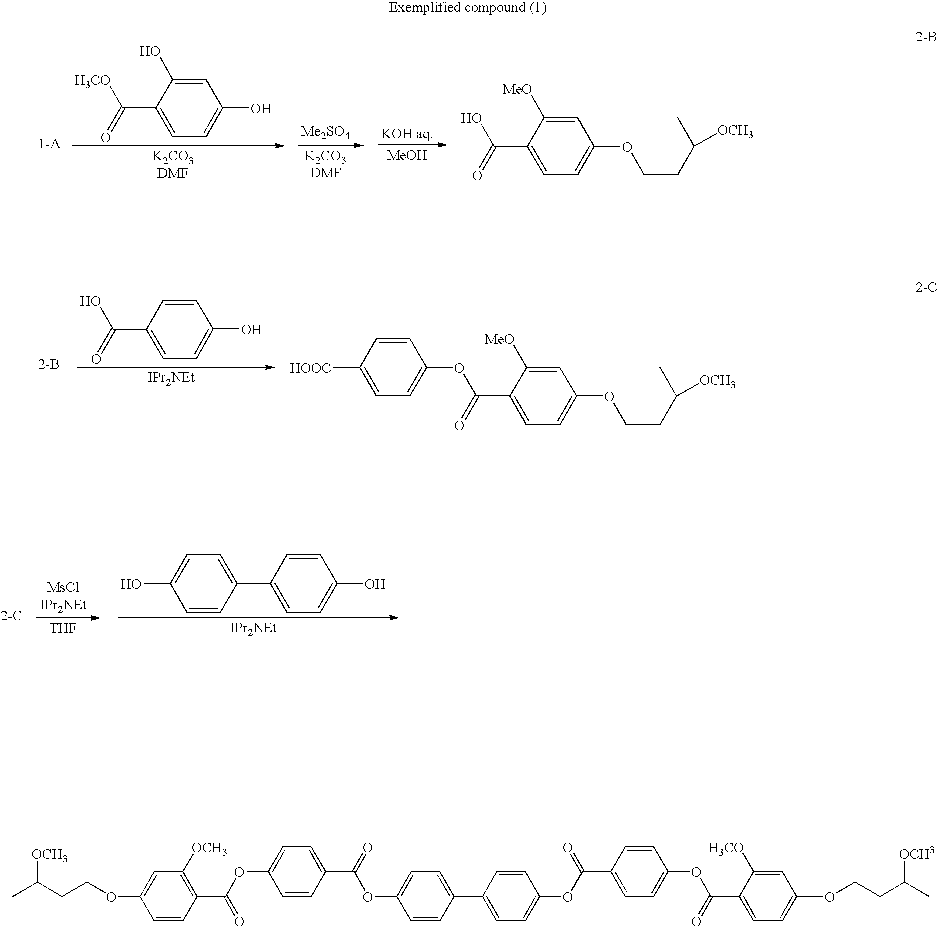

### [Production of Intermediate (2-B)]

In 500 ml of DMF, were added 67.3 g of methyl 2,4-dihydroxybenzoate, 100 g of the intermediate (1-A) obtained in Example 1, and 137.5 g of potassium carbonate, and the resultant mixture was stirred under heating for 6 hours on a 90° C. hot bath. Then, to the mixture, 75.9 ml of dimethyl sulfate and 137 g of potassium carbonate were added, followed by stirring under heating for 10 hours. After the reaction was finished, 500 ml of ethyl acetate was added to the reaction solution, and inorganic salts were then separated off by filtration under reduced pressure. Water was added to the filtrate, and the organic phase was extracted twice with ethyl acetate. The organic phase was washed with 1-N aqueous hydrochloric acid, water, and saturated brine, in this order, and then dried over magnesium carbonate, and then the solvents were distilled off under reduced pressure. The residue was purified on silica gel column chromatography (developing solvent, ethyl acetate/n-hexane=1/10), to obtain 70 g of an intermediate methyl ester body as an oily substance (yield 59%). This methyl ester body was dissolved in 150 ml of methanol and 143 ml of an aqueous 5-N potassium hydroxide solution, and the resultant solution was refluxed under heating for 2 hours. Then, the resultant solution was added into 59.3 ml of a mixture solution containing 500 ml of water and 59.3 ml of 12-N aqueous hydrochloric acid. Then, thereto 500 ml of ethyl acetate was added, to separate the organic phase. The organic phase was washed with water and saturated brine in this order, and then dried over magnesium sulfate, and then the solvents were distilled off under reduced pressure, to thus obtain 66 g of an intermediate (2-B) as an oily substance.

### [Production of Intermediate (2-C)]

Were heated 25.4 g of the intermediate (2-B), 100 ml of toluene, and 0.1 ml of dimethylformamide, to 70° C., and then to the resultant mixture, 8.03 ml of thionyl chloride was slowly added dropwise. After the dropwise addition was finished, the mixture was stirred under heating at 70° C. for 1 hour. Then, to the reaction solution, a solution obtained by dissolving 13.81 g of 4-hydroxybenzoic acid in 20 ml of dimethylformamide was added, followed by conducting the reaction at 70° C. for further 2 hours. After the reaction was finished, the reaction solution was cooled to the room temperature, and then, ethyl acetate and water were added thereto, to separate the organic phase. The organic phase was washed with 1-N aqueous hydrochloric acid, water and saturated brine, in this order, and then dried over magnesium carbonate, and then the solvents were distilled off under reduced pressure. The residue was purified on silica gel column chromatography (developing solvent, methylene chloride/methanol=20/1), and dispersed in n-hexane, followed by filtration under reduced pressure, to obtain 21 g of an intermediate (2-C) as a white solid.

### [Production of Exemplified Compound (1)]

To 80 ml of a tetrahydrofuran solution containing 6 g (16 mmol) of the intermediate (2-C), added was 1.2 ml (16 mmol) of methansulfonic acid chloride, under ice-cooling, and to the resultant mixture, added slowly, dropwise, was 2.8 ml (16 mmol) of N,N-diisopropylethylamine. After the resultant mixture was stirred for one hour, to the mixture, 5.6 ml (32 mmol) of N,N-diisopropylethylamine was added, and then 30 ml of a tetrahydrofuran solution containing 1.36 g of 4,4′-biphenol was added dropwise. Then, to the mixture, 5 ml of a tetrahydrofuran (THF) solution containing 0.1 g of N,N-dimethylaminopyridine was added dropwise, followed by stirring under ice-cooling for one hour, then heating the reaction solution to the room temperature, and further stirring for 6 hours. The reaction solution was added dropwise to 1 L of methanol, and the thus-produced crystals were collected by filtration, and dried, to obtain 5.0 g of the Exemplified compound (1).

The compound was identified by ¹H-NMR.

¹H-NMR (CDCl₃): δ 1.25 (d, 6H), 2.00 (m, 4H), 3.35 (s, 6H), 3.60 (m, 2H) 3.95 (s, 6H), 4.15 (m, 4H), 6.55 (m, 4H), 7.30 (d, 4H), 7.40 (d, 4H), 7.65 (d, 4H), 8.10 (d, 2H), 8.30 (d, 4H)

### Example 3

### Production of Exemplified Compound (36)

The Exemplified compound (36) was produced, according to the following scheme.

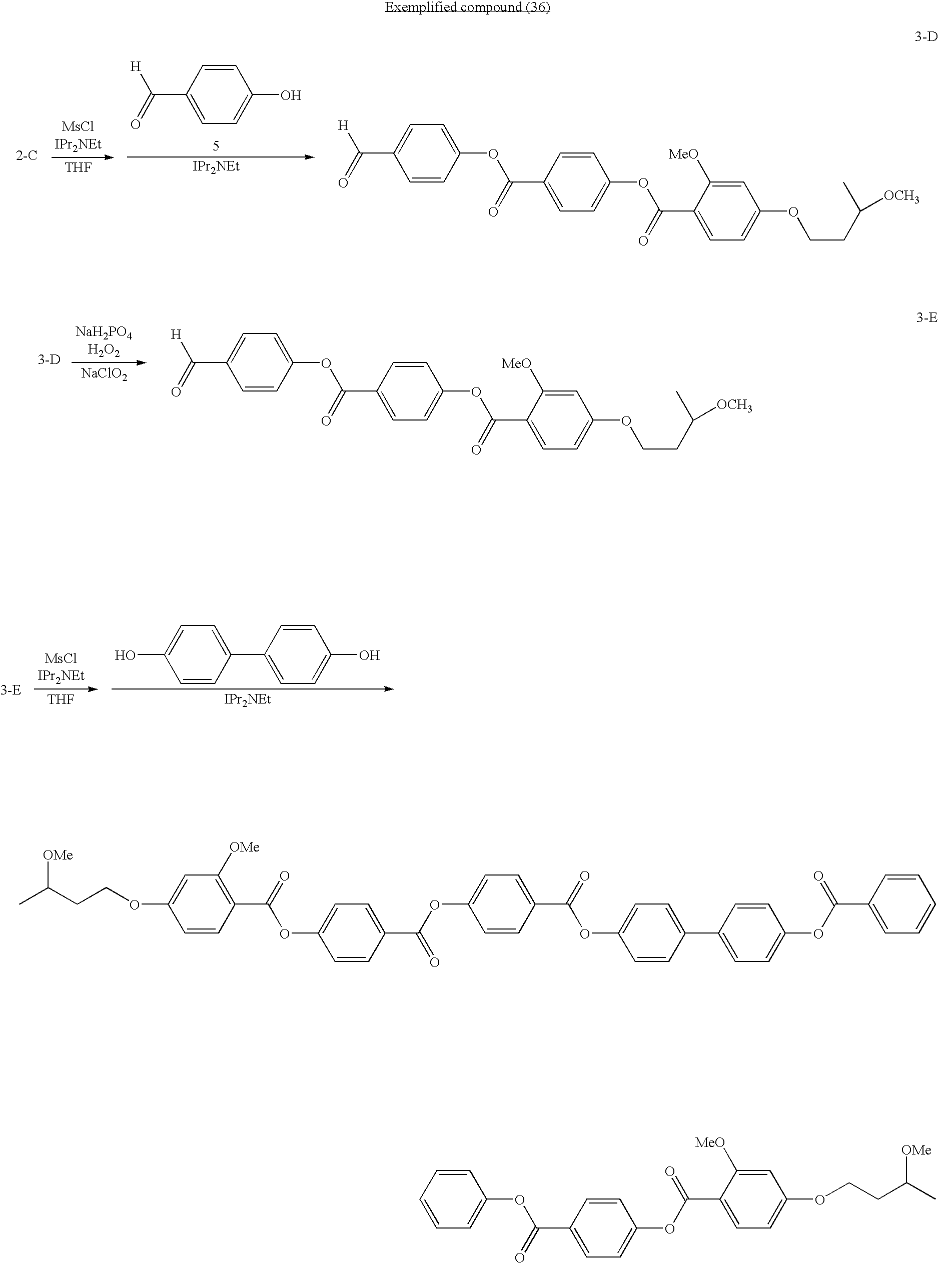

### [Production of Intermediate (3-D)]

To 150 ml of a tetrahydrofuran solution containing 15 g (0.04 mol) of the intermediate (2-C) obtained in Example 2, added was 3.1 ml (0.04 mol) of methansulfonic acid chloride, under ice-cooling, and to the resultant mixture, added slowly, dropwise, was 7.8 ml (0.045 mol) of N,N-diisopropylethylamine (IPr₂NEt). After the resultant mixture was stirred for one hour, to the mixture, 10 ml of a tetrahydrofuran solution containing 5.4 g (0.045 mol) of parahydroxybenzaldehyde, and 7.8 ml (0.045 mol) of N,N-diisopropylethylamine were added, dropwise. Then, to the mixture, 5 ml of a tetrahydrofuran solution containing 0.05 g of N,N-dimethylaminopyridine was added dropwise, followed by stirring under ice-cooling for one hour, then raising to the room temperature, and further stirring for 6 hours. To the reaction solution, water was added, to separate the solution, and the separated organic phase was washed with water, 1-N aqueous hydrochloric acid, and water, in this order. The resulting organic phase was dried over magnesium sulfate, and the solvent was distilled off under reduced pressure, to obtain 20 g of a crude intermediate (3-d).

### [Production of Intermediate (3-E)]

Seventy two milliliters of an acetonitrile solution containing 20 g (0.041 mol) of the intermediate (3-D) was cooled to 10° C., and thereto 7 ml of an aqueous solution containing 1.3 g (0.008 mol) of sodium dihydrogenphosphate was added dropwise, and then 7.1 ml (0.062 mol) of a 30% aqueous hydrogen peroxide was added dropwise. To the mixture, 35 ml of an aqueous solution containing 4.9 g of sodium chlorite was slowly added dropwise. The temperature of the reaction system was raised to 50° C., followed by stirring for 3 hours. The completion of the reaction was confirmed by TLC, and then the reaction system was cooled to 10° C. The reaction solution was slowly added dropwise to 1.5 L of 1-N aqueous hydrochloric acid cooled to 5° C. The thus-produced crystals were collected by filtration, and dried, to obtain 20 g of an intermediate (3-E).

### [Production of Exemplified Compound (36)]

To 150 ml of a tetrahydrofuran solution containing 6 g (12 mmol) of the intermediate (3-E), added was 0.94 ml (12 mmol) of methansulfonic acid chloride, under ice-cooling, and to the resultant mixture, added slowly, dropwise, was 2.2 ml (12 mmol) of N,N-diisopropylethylamine. After the resultant mixture was stirred for one hour, to the mixture, 4.4 ml (24 mmol) of N,N-diisopropylethylamine was added, and then 30 ml of a tetrahydrofuran solution containing 1.03 g of 4,4′-biphenol was added dropwise. Then, to the mixture, 5 ml of a tetrahydrofuran (THF) solution containing 0.1 g of N,N-dimethylaminopyridine was added dropwise, followed by stirring under ice-cooling for one hour, then heating the reaction solution to the room temperature, and further stirring for 6 hours. The reaction solution was added dropwise to 1 L of methanol, and the thus-produced crystals were collected by filtration, and dried, to obtain 4.8 g of the Exemplified compound (36).

The compound was identified by ¹H-NMR.

¹H-NMR (CDCl₃): δ 1.25 (d, 6H), 2.00 (m, 4H), 3.35 (s, 6H), 3.60 (m, 2H) 3.95 (s, 6H), 4.15 (m, 4H), 6.55 (m, 4H), 7.30 (d, 4H), 7.40 (d, 8H), 7.65 (d, 4H), 8.10 (d, 2H), 8.30 (d, 8H)

### Example 4

### Production of Exemplified Compound (26)

The Exemplified compound (26) was produced, according to the following scheme.

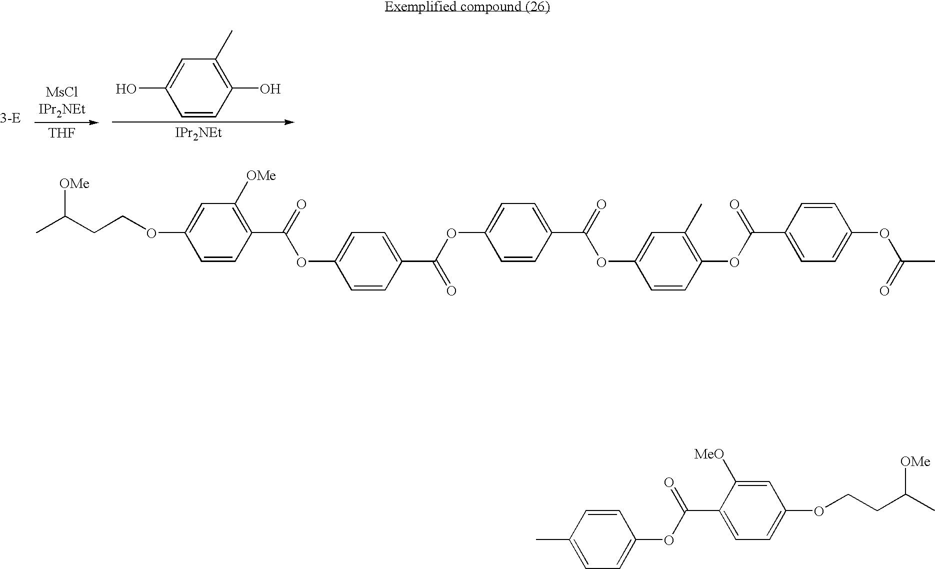

To 150 ml of a tetrahydrofuran solution containing 6 g (12 mmol) of the intermediate (3-E) obtained in Example 3, added was 0.94 ml (12 mmol) of methansulfonic acid chloride, under ice-cooling, and to the resultant mixture, added slowly, dropwise, was 2.2 ml (12 mmol) of N,N-diisopropylethylamine. After the resultant mixture was stirred for one hour, to the mixture, 4.4 ml (24 mmol) of N,N-diisopropylethylamine was added, and then 30 ml of a tetrahydrofuran solution containing 0.69 g of methylhydroquinone was added dropwise. Then, to the mixture, 5 ml of a tetrahydrofuran (THF) solution containing 0.1 g of N,N-dimethylaminopyridine was added dropwise, followed by stirring under ice-cooling for one hour, then heating the reaction solution to the room temperature, and further stirring for 6 hours. The reaction solution was added dropwise to 1 L of methanol, and the thus-produced crystals were collected by filtration, and dried, to obtain 4.4 g of the Exemplified compound (26).

The compound was identified by ¹H-NMR.

¹H-NMR (CDCl₃): δ 1.25 (d, 6H), 2.00 (m, 4H), 3.35 (s, 6H), 3.60 (m, 2H), 3.95 (s, 6H), 4.15 (m, 4H), 6.55 (m, 4H), 7.15 (m, 3H), 7.40 (m, 8H), 8.10 (m, 2H), 8.30 (m, 8H)

### Example 5

A test was conducted to evaluate the solubility of the compound represented by formula (1) or (2), which can be used in the present invention, in a halogen-containing mixture solution. That is, any of the Exemplified compound (1) or (11), or an analogous compound (1), which is a compound for comparison, was dissolved in a mixture solvent of 87 parts by mass of methylene chloride and 13 parts by mass of methanol, to determine solubility of each compound in the mixture solvent. The results are shown in Table 1.

TABLE 1

Analogous compound (1)

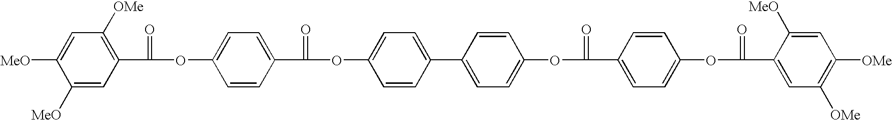

| | | Methylene chloride:Methanol = 87:13 |
|---|---|---|
| No. | Additive | Solubility |
| 1 | Analogous compound | 1% |
| 2 | Exemplified compound (1) | 30% |
| 3 | Exemplified compound (11) | 15% |

As is apparent from the results shown in Table 1, it is understood that the compound represented by formula (1) or (2) that can be used in the present invention, exhibited remarkably higher solubility in the mixture solvent of: (methylene chloride:methanol=87:13 (mass ratio)), than the analogous compound.

### Example 6

The following components of a cellulose acetate solution composition were charged into a mixing tank, followed by stirring under heating, to dissolve the components each other. Thus, a cellulose acetate solution was prepared.

(Composition of a cellulose acetate solution)

| | |
|---|---|
| Cellulose acetate (acetylation degree 60.9%) | 100 mass parts |
| Triphenyl phosphate (plasticizer) | 7.8 mass parts |
| Biphenyl diphenyl phosphate (plasticizer) | 3.9 mass parts |
| Methylene chloride (first solvent) | 318 mass parts |
| Methanol (second solvent) | 47 mass parts |

To another mixing tank, 6 mass parts of the Exemplified compound (1) or (11) or Compound 1 for comparison, 87 mass parts of methylene chloride, and 13 mass parts of methanol were charged with, followed by stirring under heating, to prepare a retardation controlling agent solution, respectively.

Then, 36 mass parts of the retardation controlling (increasing) agent solution was mixed with 474 mass parts of the cellulose acetate solution, and the resultant mixture was thoroughly stirred, to prepare a dope. The retardation controlling agents each were added in an amount in terms of mass parts, as shown in Table 2, to 100 mass parts of the cellulose acetate.

The thus-obtained dope was cast, using a band casting machine. The resultant film in which the residual solvent amount was 15 mass %, was laterally oriented, using a tenter, under the conditions of 130° C., at an orientation ratio of 20%, to prepare a cellulose acetate film (thickness 92 μm). With respect to the thus-produced cellulose acetate films (optical compensation sheets), the Re retardation values and Rth retardation values at wavelength 633 nm were measured, using an ellipsometer (M-150, trade name, manufactured by JASCO Corporation). The results are shown in Table 2.

### <Measurement of Rth and Re>

The Rth and Re values were obtained, by utilizing the film thickness, the refractive indexes in the plane, each of which was measured at wavelength 632.8 nm, by using an ellipsometer (AEP-100, trade name, manufactured by Shimadzu Corporation), according to the following equations:

Re=(nx−ny)×d

Rth={(nx+ny)/2−nz}×d

in which,

nx: Refractive index in the direction of the slow phase axis (i.e. the direction in which the refractive index would be maximum);

ny: Refractive index in the direction perpendicular to the slow phase axis

nz: Refractive index in the direction of the thickness

d: Thickness of the film (unit: nm)

TABLE 2

| | | Amount to | | | | |
|---|---|---|---|---|---|---|
| Sample | | be added | Re | Rth | | |
| No | Additive | (mass parts) | (nm) | (nm) | Re/Rth | Remarks |
| 1 | None | 0 | 2 | 50 | 0.04 | Comparative example |
| 2 | Compound 1 for comparison | 2 | 20 | 115 | 0.17 | Comparative example |
| 3 | Compound 1 for comparison | 5 | 38 | 180 | 0.21 | Comparative example |
| 4 | Exemplified compound (1) | 2 | 58 | 115 | 0.50 | This invention |
| 5 | Exemplified compound (1) | 5 | 180 | 250 | 0.72 | This invention |
| 6 | Exemplified compound (11) | 2 | 54 | 125 | 0.43 | This invention |
| 7 | Exemplified compound (11) | 5 | 165 | 260 | 0.63 | This invention |

Compound 1 for comparison (Compound described in JP-A-2003-344655)

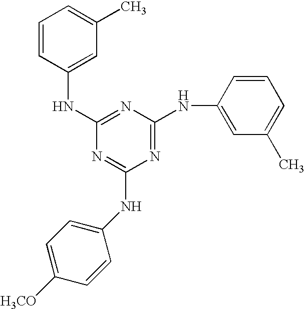

As is apparent from the results shown in Table 2, the film containing no Compound I for comparison, the film containing 2 parts by mass of the Compound 1 for comparison, and the film containing 5 parts by mass of the Compound 1 for comparison each fulfill the following expression.

Rth=Re×3.6+43 (Expression 1)

Specifically, the film containing the Compound 1 for comparison developed Re but did not much change Rth upon stretching, and thus a film fulfilling the optical performances in the region satisfying the following expression, can be produced, by adding the Compound 1 for comparison.

Rth≧Re×3.6+43 (Expression 2)

Contrary to the above, it is also found that the film of the present invention containing the compound represented by formula (1) or (2), i.e. Exemplified compound (1) or (11), can give a novel cellulose film, which is capable of developing a remarkably larger Re, i.e., in the region where the following expression is fulfilled, which is not attained by the film containing the Compound 1 for comparison.

Rth<Re×3.6+43

Specifically, as is apparent from the results shown in Table 2, it is found that, by utilizing the compound of the formula (1) or (2) according to the present invention, it is possible to produce a novel cellulose film having excellent optical performances in the region of the higher Re developing property, which are not attained by a known disk-like compound (e.g. Compound 1 for comparison).

Further, as is apparent from the results shown in Table 2, using the exemplified compound of the present invention is also advantageous, from the viewpoint of reduction of the production cost, since the compound of the present invention can exhibit the same level of Re developing property as the compound for comparison even if the amount of the exemplified compound to be added is smaller. Further, as can be seen from the fact that the value of Re/Rth of the exemplified compound is remarkably larger than that of the compound for comparison, it is understood that the exemplified compound of the present invention is a compound that attains a quite high Re and a quite low Rth, which are preferable.

Further, it is understood that, also taking the results of Example 5 into consideration, the cellulose compound composition of the present invention in which the compound represented by formula (1) or (2) is contained, can exhibit excellent solubility in a halogen-containing mixture solution.

Having described our invention as related to the present embodiments, it is our intention that the invention not be limited by any of the details of the description, unless otherwise specified, but rather be construed broadly within its spirit and scope as set out in the accompanying claims.

1. A cellulose compound composition, comprising at least one compound represented by formula (1): 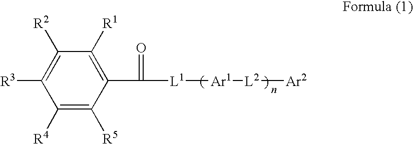
wherein R¹, R², R³, R⁴ and R⁵ each independently represent a hydrogen atom or a substituent, and any one of R¹, R², R³, R⁴ and R⁵ represents a group represented by -L³-Q¹, in which Q¹ represents a group represented by formula Q1: 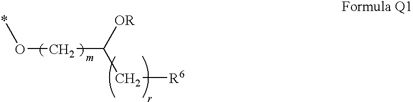
in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;
L¹, L² and L³ each independently represent a single bond or a divalent linking group, Ar¹ represents an arylene group or an aromatic heterocyclic group, Ar² represents an aryl group or an aromatic heterocyclic group, and n represents an integer of 0 or more, in which two or more L²s may be the same or different from each other and two or more Ar¹s may be the same or different from each other.

2. The cellulose compound composition according to claim 1, wherein R³ in formula (1) is the group represented by -L³-Q¹.

3. The cellulose compound composition according to claim 1, wherein the compound represented by formula (1) is a compound represented by formula (2): 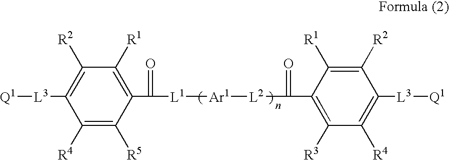
wherein R¹, R², R³, R⁴ and R⁵ each independently represent a hydrogen atom or a substituent, and Q¹ represents a group represented by formula Q1: 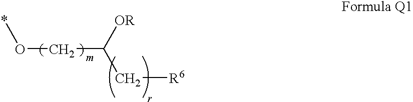
in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;
L¹, L² and L³ each independently represent a single bond or a divalent linking group, Ar¹ represents an arylene group or an aromatic heterocyclic group, and n represents an integer of 1 or more, in which two or more L²s may be the same or different from each other and two or more Ar¹s may be the same or different from each other.

4. The cellulose compound composition according to claim 1, wherein a cellulose acylate is contained as the cellulose compound.

5. A cellulose compound film, which is composed of the cellulose compound composition according to claim 1.

6. A compound, represented by formula (1): 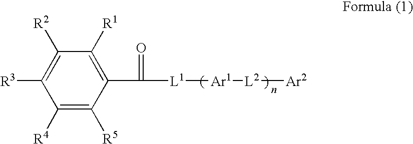
wherein R¹, R², R³, R⁴ and R⁵ each independently represent a hydrogen atom or a substituent, and any one of R¹, R², R³, R⁴ and R⁵ represents a group represented by -L³-Q¹, in which Q¹ represents a group represented by formula Q1: 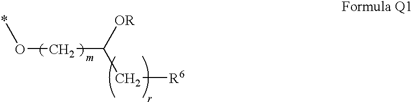
in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;
L¹, L² and L³ each independently represent a single bond or a divalent linking group, Ar¹ represents an arylene group or an aromatic heterocyclic group, Ar² represents an aryl group or an aromatic heterocyclic group, and n represents an integer of 0 or more, in which two or more L²s may be the same or different from each other and two or more Ar¹s may be the same or different from each other.

7. A compound, represented by formula (2): 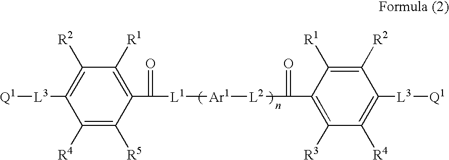
wherein R¹, R², R³, R⁴ and R⁵ each independently represent a hydrogen atom or a substituent, and Q¹ represents a group represented by formula Q1: 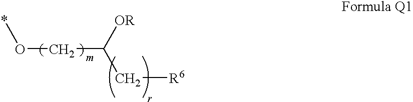
in which * represents a bonding hand for bonding with L³, R represents a hydrogen atom or an alkyl group having one or more carbon atoms, m denotes an integer of 0 or more, r represents an integer of 0 or more, R⁶ represents a hydrogen atom or a substituent;
L¹, L² and L³ each independently represent a single bond or a divalent linking group, Ar¹ represents an arylene group or an aromatic heterocyclic group, and n represents an integer of 1 or more, in which two or more L²s may be the same or different from each other and two or more Ar¹s may be the same or different from each other.
